(19) United States
(12) Patent Application Publication
(10) Pub. No.: US 2015/0247204 A1
(43) Pub. Date: Sep. 3, 2015

(54) DIFFERENTIAL DIAGNOSIS OF HEPATIC NEOPLASMS

(71) Applicants: The General Hopital Corporation, Boston, MA (US); Affymetrix, Inc., Santa Clara, CA (US)

(72) Inventors: Vikram Deshpande, Belmont, MA (US); Manoj Gandhi, Dublin, CA (US); Quan Nguyen, San Ramon, CA (US); Yunqing MA, San Jose, CA (US); David T. Ting, Dover, MA (US); Miguel Rivera, Cambridge, MA (US)

(21) Appl. No.: 14/616,297

(22) Filed: Feb. 6, 2015

Related U.S. Application Data
(60) Provisional application No. 62/080,594, filed on Nov. 17, 2014.
(60) Provisional application No. 61/937,336, filed on Feb. 7, 2014.

(51) Int. Cl. C12Q 1/68

(52) U.S. Cl. CPC C12Q 1/6886; C12Q 1/6809; C12Q 2600/158; C12Q 2600/16; C12Q 2600/112

(57) ABSTRACT

Methods for making a differential diagnosis of hepatic neoplasms, e.g., tumors, and for identifying metastatic tumors of hepatic origin, based on detection of levels of albumin mRNA. The methods can also be used to select treatments or guide treatment decisions.

### CLAIM OF PRIORITY

This application claims the benefit of U.S. Provisional Patent Application Ser. Nos. 61/937,336, filed on Feb. 7, 2014, and 62/080,594, filed on Nov. 17, 2014. The entire contents of the foregoing are hereby incorporated by reference.

### TECHNICAL FIELD

Described are methods for making a differential diagnosis of hepatic neoplasms, e.g., tumors, based on levels of albumin mRNA, and for identifying metastatic tumors of hepatic origin.

### BACKGROUND

Hepatocellular carcinoma (HCC) is the third most common cause of cancer related mortality worldwide. The immunohistochemical diagnosis of poorly differentiated and undifferentiated hepatocellular carcinoma has traditionally been difficult because of a lack of a reliable marker of hepatocellular differentiation (Chan and Yeh, Clinics in Liver Disease 14, 687-703 (2010); Kakar et al., Arch Pathol Lab Med. 2007 November; 131(11): 1648-54). Alpha fetoprotein (AFP) and polyclonal carcinoembryonic antigen (CEA) were the first of many markers of hepatocellular differentiation that have emerged in the last four decades. However, sensitivity of these markers is low—AFP ranges from 30% to 50% and polyclonal CEA from 60% to 90% (Kakar et al., American Journal of Clinical Pathology 119, 361-366 (2003)). Background staining can make these markers difficult to interpret, and furthermore, these markers are more likely to be negative in poorly differentiated and undifferentiated carcinomas (Chan and Yeh, Clinics in Liver Disease 14, 687-703 (2010); Kakar et al., Arch Pathol Lab Med. 2007 November; 131(11):1648-54). Hep Par 1, a sensitive marker of hepatocellular differentiation represented a significant advance over the prior assays (Chan and Yeh, Clinics in Liver Disease 14, 687-703 (2010); Kakar et al., American Journal of Clinical Pathology 119, 361-366 (2003); Chu et al., Am J Surg Pathol 26, 978-988 (2002)). Furthermore, the bright granular cytoplasmic reactivity made interpretation relatively easy and thus it quickly emerged as definitive marker of hepatocellular differentiation. With its widespread usage two conspicuous failings emerged (Fan et al., Mod Pathol 16, 137-144 (2003)), as follows: 1. A significant percentage of gastric, esophageal and lung adenocarcinomas showed strong reactivity, a particularly troubling finding since these tumors frequently metastasize to the liver, 2. The sensitivity for poorly differentiated hepatocellular carcinomas was significantly lower than for well-differentiated tumors, thus limiting its diagnostic value.

Cholangiocarcinomas (CCAs) are classified based on their anatomic location as follows: (1) intrahepatic cholangiocarcinoma (IHCC), (2) perihilar cholangiocarcinoma, or (3) distal cholangiocarcinoma (Whithaus et al., Arch Pathol Lab Med 136:155-162, 2012; Blechacz et al., Nat Rev Gastroenterol Hepatol 8:512-522,2011; Razumilava et al., Clin Gastroenterol Hepatol 11:13-21.e1-quiz e3-4, 2013). The latter two entities are often referred to as bile duct carcinoma (Razumilava et al., Clin Gastroenterol Hepatol 11:13-21.e1-quiz e3-4, 2013). Though they share some similarities, IHCC has different clinical behavior than bile duct cancer, and therefore, suggests distinguishing molecular features between these entities. IHCC is the second-most common primary liver cancer; its incidence has increased by 22% between 1979 and 2004 (Whithaus et al., Arch Pathol Lab Med 136:155-162, 2012; Blechacz et al., Nat Rev Gastroenterol Hepatol 8:512-522, 2011; Everhart et al., Gastroenterology 136:1134-1144, 2009). IHCC generally presents as a solitary, and less often multiple intrahepatic lesions (Khan et al., Gut 61:1657-1669, 2012; De Jong et al., Journal of Clinical Oncology 29:3140-3145,2011; Hong et al., Surgery 146:250-257, 2009). Only subtle histopathological differences exist between IHCC and metastatic adenocarcinoma to the liver, such that the majority of IHCCs cannot be distinguished from a metastatic adenocarcinoma to the liver with a high degree of certainty. Immunohistochemistry may assist in this distinction—e.g., reactivity for transcriptional factors such as TTF-1 support a metastatic pulmonary adenocarcinoma. Nevertheless, infidelity among these transcriptional factors is well recognized and the sensitivity of these assays is low (Whithaus et al., Arch Pathol Lab Med 136:155-162, 2012; Hainsworth et al., Journal of Clinical Oncology 31:217-223,2013). Pathologists often resort to evaluating keratin profiles: cholangiocarcinomas are typically positive for keratin 7, and keratin 19, and occasionally for keratin 20. Unfortunately, a majority of metastatic adenocarcinomas share this keratin 7+, keratin 19+ and keratin 20− profile, and in this scenario immunohistochemistry is seldom precisely diagnostic.

With this inability to distinguish primary liver adenocarcinoma from a metastatic neoplasm, the current clinical paradigm demands an exhaustive evaluation to exclude a potential primary adenocarcinoma at other sites (Khan et al., Gut 61:1657-1669, 2012). These investigations often include various imaging studies (e.g., cervical pap smear, mammogram, computed tomography, magnetic resonance imaging, and positron emission tomography) as well as upper and lower endoscopic evaluation. Blood work is also not very helpful given the commonly used markers (i.e. CA19-9, CEA, and CA125) are not specific to IHCC. In patients with concurrent hepatic and non-hepatic disease, it is virtually impossible to exclude an IHCC. These workups could be costly and time-consuming leading to a delayed diagnosis and treatment with implications for eligibility for and stratification of clinical trials.

### SUMMARY

Malignant neoplasms in the liver can be either primary (originating within the liver) or metastatic (spread from other organs). Malignant primary liver neoplasms include tumors derived from hepatocytes, known as Hepatocellular Carcinoma (HCC); malignant tumors derived from the bile ducts within the liver, known as Intrahepatic Cholangiocarcinoma (IHCC); and bile duct adenomas (BDAs, also sometimes called peribiliary gland hamartomas), which is a benign tumor also of the bile duct. The liver is one of the most common sites of involvement in the metastatic spread of cancers. The most common primary sources are those of colon, breast, lung and pancreas, kidney, melanoma, leukemia and lymphoma.

Primary and metastatic malignant neoplasms of the liver demonstrate a wide spectrum of histologic patterns. In many instances, it is difficult to distinguish malignant primary liver tumors from metastatic liver lesions using routine light microscopy. This distinction is important because patients with primary liver tumors are treated differently from patients with metastatic liver disease. Primary liver tumors are treated with surgical resection, liver transplantation or targeted therapy with tyrosine Kinase inhibitors. On the contrary, patients with metastatic liver disease are treated with palliative treatment or systemic chemotherapy.

Albumin, a protein synthesized by hepatocytes, was first proposed as a marker of hepatocellular differentiation in the late 1980s (Kojior et al., Lab Invest 44, 221-226 (1981)). However, immunohistochemical detection of albumin in FFPE tissues has proved difficult primarily because of its ubiquitous presence as a secreted protein and results in staining patterns that are difficult to interpret and lacks the desired sensitivity and specificity. Detecting mRNA instead of protein has been attempted (see, e.g., Kaker et al., American Journal of Clinical Pathology 119, 361-366 (2003); Oliveira et al., Am J Surg Pathol 24, 177-182 (2000); Krishna et al., Am J Surg Pathol 21, 147-152 (1997); Murray et al., J Clin Pathol 45, 21-24 (1992); D'Errico et al., Hum Pathol 27, 599-604 (1996); D'Errico et al., Diagn Mol Pathol 7, 289-294 (1998)). However, in spite of the fact that albumin has the potential to be a highly sensitive and specific marker for hepatocellular carcinoma, it has not found widespread use in the diagnostic laboratory, as consequence of two unresolved problems: 1. The lability of mRNA compared to proteins and 2. The lack of a robust and sensitive platform for the detection of RNA in situ.

As shown herein, albumin is synthesized almost exclusively by cancers of liver origin; thus, it can be used as a key marker to diagnose these malignancies. A survey of gene expression across different cancer types indicated that albumin expression is highly restricted to the liver among normal tissues and is selectively present in hepatocellular carcinoma and IHCC, as well as in BDA, as compared to other tumor types. As shown herein, expression of mRNA coding for secreted proteins such as albumin can be reliably detected in paraffin embedded tissue using branched DNA analysis. The present experiments show that cancers of hepatic origin are positive for albumin, a signature absent in non-hepatic lesions as well as perihilar and bile duct carcinomas, distinguishing BDA, IHCC and HCC from a variety of metastatic adenocarcinomas.

Provided herein are methods for determining the origin of a tumor in a subject. The methods include contacting a sample comprising cells or tissue from the tumor with a plurality of polynucleotide probes that bind specifically to albumin mRNA in situ; detecting binding of the probes to albumin mRNA, and either identifying a sample in which the probes bind to albumin mRNA as a tumor of hepatic origin, or identifying a sample in which the probes do not bind to albumin mRNA as a tumor of nonhepatic origin.

Also provided herein are methods for selecting a treatment for a subject who has a tumor. The methods include contacting a sample comprising cells or tissue from the tumor with a plurality of polynucleotide probes that bind specifically to albumin mRNA in situ; detecting binding of the probes to albumin mRNA, and either:
identifying a sample in which the probes bind to albumin mRNA as a tumor of hepatic origin, and selecting for the subject a treatment for a hepatic tumor; or
identifying a sample in which the probes do not bind to albumin mRNA as a tumor of nonhepatic origin; determining the tissue of origin of the tumor; and selecting for the subject a treatment for a cancer of the tissue of origin.

In addition, provided herein are methods for treating a subject who has a tumor. The methods include contacting a sample comprising cells or tissue from the tumor with a plurality of polynucleotide probes that bind specifically to albumin mRNA in situ; detecting binding of the probes to albumin mRNA, and either identifying a sample in which the probes bind to albumin mRNA as a tumor of hepatic origin, and administering to the subject a treatment for a hepatic tumor; or identifying a sample in which the probes do not bind to albumin mRNA as a tumor of nonhepatic origin; determining the tissue of origin of the tumor; and administering to the subject a treatment for a cancer of the tissue of origin.

Also provided herein are methods for differential diagnosis between metastatic liver disease and a primary tumor of hepatic origin in a subject who has a tumor. The methods include contacting a sample comprising tissue from the tumor with a plurality of polynucleotide probes that bind specifically to albumin mRNA in situ; detecting binding of the probes to albumin mRNA, and either identifying a sample in which the probes bind to albumin mRNA as a tumor of hepatic origin, or identifying a sample in which the probes do not bind to albumin mRNA as a tumor of nonhepatic origin.

In some embodiments, the methods include determining whether a tumor of hepatic origin is Hepatocellular Carcinoma (HCC) or Intrahepatic Cholangiocarcinoma (IHCC), or whether a tumor is an HCC, IHCC, or bile duct adenoma (BDA).

In some embodiments, whether the tumor is HCC or IHC or BDA is determined based on morphology of the tumor cells in the sample, e.g., determined by histopathological analysis, e.g., staining the sample with hematoxylin and eosin and examining the sample using light microscopy, wherein a trabecular arrangement of tumor cells resembling normal hepatocytes indicates the presence of HCC and a tubulo-glandular arrangement of tumor cells resembling adenocarcinoma indicates the presence of IHCC.

In some embodiments, whether the tumor is HCC or IHC or BDA is determined based on morphology of the tumor cells in the sample, e.g., determined by histopathological analysis, e.g., staining the sample with hematoxylin and eosin and examining the sample using light microscopy, wherein a trabecular arrangement of tumor cells resembling normal hepatocytes indicates the presence of HCC, a tubulo-glandular arrangement of tumor cells resembling adenocarcinoma indicates the presence of IHCC, and tumor architecture resembling adenoma with a well differentiated glandular pattern indicates the presence of BDA.

In some embodiments, the methods include one or more of:

identifying a sample in which the probes bind to albumin mRNA, and wherein there is a gradient of albumin mRNA present in hepatoctyes with low to moderate expression in Zones 1 and 3 and high expression in Zone 2, as comprising normal liver; identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma; identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma; and identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease.

In some embodiments, the methods include one or more of:

identifying a sample in which the probes bind to albumin mRNA, and wherein there is a gradient of albumin mRNA present in hepatoctyes with low to moderate expression in Zones 1 and 3 and high expression in Zone 2, as comprising normal liver; identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma; identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma; identifying a sample in which the probes bind to albumin mRNA, there are low levels of albumin mRNA expression, and tumor architecture resembling adenoma with a well differentiated glandular pattern as comprising BDA; and identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease.

In some embodiments, the methods include one or more of: identifying a sample in which the probes bind to albumin mRNA, and wherein there is a gradient of albumin mRNA present in hepatoctyes with low to moderate expression in Zones 1 and 3 and high expression in Zone 2, as comprising normal liver, and not treating the subject; identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma (HCC), and selecting a treatment for HCC for the subject; identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma (IHCC), and selecting a treatment for IHCC for the subject; and identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease, and selecting a treatment for the primary cancer for the subject.

In some embodiments, the methods include one or more of: identifying a sample in which the probes bind to albumin mRNA, and wherein there is a gradient of albumin mRNA present in hepatoctyes with low to moderate expression in Zones 1 and 3 and high expression in Zone 2, as comprising normal liver, and not treating the subject; identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma (HCC), and selecting a treatment for HCC for the subject; identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma (IHCC), and selecting a treatment for IHCC for the subject; identifying a sample in which the probes bind to albumin mRNA, there are low levels of albumin mRNA expression, and tumor architecture resembling adenoma with a well differentiated glandular pattern as comprising BDA, and selecting a treatment for BDA for the subject; and identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease, and selecting a treatment for the primary cancer for the subject.

In some embodiments, the methods include one or more of: identifying a sample in which the probes bind to albumin mRNA, and wherein there is a gradient of albumin mRNA present in hepatoctyes with low to moderate expression in Zones 1 and 3 and high expression in Zone 2, as comprising normal liver, and not treating the subject; identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma (HCC), and administering a treatment for HCC to the subject; identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma (IHCC), and administering a treatment for IHCC to the subject; and identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease, and administering a treatment for the primary cancer to the subject.

In some embodiments, the methods include one or more of: identifying a sample in which the probes bind to albumin mRNA, and wherein there is a gradient of albumin mRNA present in hepatoctyes with low to moderate expression in Zones 1 and 3 and high expression in Zone 2, as comprising normal liver, and not treating the subject; identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma (HCC), and administering a treatment for HCC to the subject; identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma (IHCC), and administering a treatment for IHCC to the subject; identifying a sample in which the probes bind to albumin mRNA, there are low levels of albumin mRNA expression, and tumor architecture resembling adenoma with a well differentiated glandular pattern as comprising BDA, and treating the subject for BDA; and identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease, and administering a treatment for the primary cancer to the subject.

In some embodiments of the methods described herein, the sample is from a tumor that is in the liver of the subject.

In some embodiments of the methods described herein, the sample is from a tumor that is not in the liver of the subject.

In some embodiments of the methods described herein, the plurality of probes comprises probes that bind to a plurality of target regions in the albumin mRNA.

In some embodiments of the methods described herein, the binding of the probes to albumin mRNA is detected using branched nucleic acid signal amplification.

In some embodiments of the methods described herein, the probes are branched DNA probes.

In some embodiments, the methods described herein include contacting the sample with a plurality of probes that comprises one or more label extender probes that bind to a plurality of target regions in the albumin mRNA; hybridizing one or more pre-amplifier probes to the one or more label extender probes; hybridizing one or more amplifier probes to the pre-amplifier probes; and hybridizing one or more label probes to the one or more amplifier probes.

In some embodiments of the methods described herein, the label probe is conjugated to alkaline phosphatase (AP), and binding of the probe is detected using fast red or fast blue as a substrate for the alkaline phosphatase.

In some embodiments of the methods described herein, the sample is a biopsy sample obtained from the subject.

In some embodiments of the methods described herein, the sample is a formaldehyde-fixed, paraffin-embedded (FFPE) clinical sample.

In some embodiments of the methods described herein, the tissue comprises a plurality of individually identifiable cells.

In some embodiments, the methods described herein include contacting a sample comprising tissue from the tumor with a plurality of polynucleotide probes that bind specifically to mRNA encoding a housekeeping gene (HKG) in situ; detecting binding of the probes to HKG mRNA, and selecting for further analysis a sample in which binding of probes to the HKG mRNA is detected, or rejecting a sample in which binding of probes do to HKG mRNA is not detected.

In some embodiments of the methods described herein, the binding of the probes to albumin mRNA or HKG mRNA is detected using branched nucleic acid signal amplification.

In some embodiments of the methods described herein, the probes are branched DNA probes.

In some embodiments, the methods described herein include contacting the sample with a plurality of probes that comprises one or more label extender probes that bind to a plurality of target regions in the albumin or HKG mRNA; hybridizing one or more pre-amplifier probes to the one or more label extender probes; hybridizing one or more amplifier probes to the pre-amplifier; and hybridizing one or more label probes to the one or more amplifier probes.

In some embodiments of the methods described herein, the label probe is conjugated to alkaline phosphatase (AP), binding of the albumin probes to albumin mRNA is detected using fast red as a substrate for the alkaline phosphatase, and binding of the HKG probes to HKG mRNA is detected using fast blue as a substrate for the alkaline phosphatase.

The invention further provides kits for performing any of the methods described herein.

The following definitions can be understood with reference to FIG. 1C. A “label extender” is a polynucleotide that is capable of hybridizing to both a nucleic acid analyte and also to at least a portion of a label probe system. A label extender typically has a first polynucleotide sequence L-1, which is complementary to a polynucleotide sequence of the nucleic acid analyte, and a second polynucleotide sequence L-2, which is complementary to a polynucleotide sequence of the label probe system (e.g., L-2 can be complementary to a polynucleotide sequence of a preamplifier, amplifier, a label probe, or the like). The label extender is preferably a single-stranded polynucleotide. Non-limiting examples of label extenders in various configurations and orientations are disclosed within, e.g., U.S. Published Patent Application No. 2012/0052498 (including but not limited to those depicted within FIGS. 10A and 10B).

A “label probe system” comprises one or more polynucleotides that collectively comprise one or more label probes which are capable of hybridizing, directly or indirectly, to one or more label extenders in order to provide a detectable signal from the labels that are associated or become associated with the label probes. Indirect hybridization of the one or more label probes to the one or more label extenders can include the use of amplifiers, or the use of both amplifiers and preamplifiers, within a particular label probe system. Label probe systems can also include two or more layers of amplifiers and/or preamplifiers to increase the size of the overall label probe system and the total number of label probes (and therefore the total number of labels that will be used) within the label probe system. The configuration of the label probe system within a particular embodiment is typically designed in the context of the overall assay, including factors such as the amount of signal required for reliable detection of the target analyte in the assay, the particular label being used and its characteristics, the number of label probes needed to provide the desired level of sensitivity, maintaining the desired balance of specificity and sensitivity of the assay, and other factors known in the art.

An “amplifier” is a polynucleotide comprising one or more polynucleotide sequences A-1 and one more polynucleotide sequences A-2. The one or more polynucleotide sequences A-1 may or may not be identical to each other, and the one or more polynucleotide sequences A-2 may or may not be identical to each other. Within label probe systems utilizing amplifiers and label probes, polynucleotide sequence A-1 is typically complementary to polynucleotide sequence L-2 of the one or more label extenders, and polynucleotide sequence A-2 is typically complementary to polynucleotide sequence LP-1 of the label probes. Within label probe systems utilizing amplifiers, preamplifiers and label probes, polynucleotide sequence A-1 is typically complementary to polynucleotide sequence P-2 of the one or more preamplifiers, and polynucleotide sequence A-2 is typically complementary to polynucleotide sequence LP-1 of the label probes. Amplifiers can be, e.g., linear or branched polynucleotides.

A “preamplifier” is a polynucleotide comprising one or more polynucleotide sequences P-1 and one or more polynucleotide sequences P-2. The one or more polynucleotide sequences P-1 may or may not be identical to each other, and the one or more polynucleotide sequences P-2 may or may not be identical to each other. When one or more preamplifiers are utilized within a label probe system, polynucleotide sequence P-1 is typically complementary to polynucleotide sequence L-2 of the label extenders, and polynucleotide sequence P-2 is typically complementary to polynucleotide sequence A-1 of the one or more amplifiers. Preamplifiers can be, e.g., linear or branched polynucleotides.

A “label probe” is a single-stranded polynucleotide that comprises a label (or optionally that is configured to bind, directly or indirectly, to a label) to directly or indirectly provide a detectable signal. The label probe typically comprises a polynucleotide sequence LP-1 that is complementary to a polynucleotide sequence within the label probe system, or alternatively to the one or more label extenders. For example, in different embodiments, label probes may hybridize to either an amplifier and/or preamplifier of the label probe system, while in other embodiments where neither an amplifier nor preamplifier is utilized, a label probe may hybridize directly to a label extender.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Labels include the use of enzymes such as alkaline phosphatase that are conjugated to an polynucleotide probe for use with an appropriate enzymatic substrate, such as fast red or fast blue, which is described within U.S. Pat. Nos. 5,780,227 and 7,033,758. Alternative enzymatic labels are also possible, such as conjugation of horseradish peroxidase to polynucleotide probes for use with 3,3′-Diaminobenzidine (DAB). Many labels are commercially available and can be used in the context of the invention.

The term “polynucleotide” encompasses any physical string of monomer units that correspond to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural (e.g., locked nucleic acids, isoG or isoC nucleotides), and can be unsubstituted, unmodified, substituted or modified. The nucleotides can be linked by phosphodiester bonds, or by phosphorothioate linkages, methylphosphonate linkages, boranophosphate linkages, or the like. Polynucleotides can additionally comprise non-nucleotide elements such as labels, quenchers, blocking groups, or the like. Polynucleotides can be, e.g., single-stranded, partially double-stranded or completely double-stranded.

The term “probe” refers to a non-analyte polynucleotide.

Two polynucleotides “hybridize” when they associate to form a stable duplex, e.g., under relevant assay conditions. Polynucleotides hybridize due to a variety of well characterized physicochemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York).

The term “complementary” refers to a polynucleotide that forms a stable duplex with its complement sequence under relevant assay conditions. Typically, two polynucleotide sequences that are complementary to each other have mismatches at less than about 20% of the bases, at less than about 10% of the bases, preferably at less than about 5% of the bases, and more preferably have no mismatches.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A: Schematic representation of an exemplary 1-plex tissue assay using a bDNA platform.

FIG. 1B: Schematic representation of an exemplary 2-plex tissue assay using a bDNA platform.

FIG. 1C: Schematic illustration of an exemplary bDNA amplification scheme.

FIGS. 2A-B: Hepatocellular carcinoma diffuse and strongly positive for albumin. A—H&E stain; B—ISH for albumin.

FIGS. 3A-F: A&B show a well differentiated cholangiocarcinoma with diffuse reactivity for albumin. C&D show a poorly differentiated cholangiocarcinoma with focal reactivity for albumin. E&F show a perihilar bile duct carcinoma that is negative for albumin. A, C, D—H&E stain; B, D, F—ISH for albumin.

FIG. 4: Exemplary diagnostic algorithm for the evaluation of hepatic neoplasms.

FIGS. 5A-1 and 5A-2: Exemplary diagnostic algorithms for the evaluation of hepatic neoplasms using a 1-plex RNA-ISH assay for albumin expression. 5A-1, Differential Diagnosis of HCC/IHCC and metastatic liver disease; 5A-2, Differential Diagnosis of HCC/IHCC/BDA and metastatic liver disease.

FIGS. 5B1-1 and 5B-2: Exemplary diagnostic algorithms for the evaluation of hepatic neoplasms using a 2-plex RNA-ISH assay for albumin and HKG expression. 5B-1, Differential Diagnosis of HCC/IHCC and metastatic liver disease; 5B-2, Differential Diagnosis of HCC/IHCC/BDA and metastatic liver disease.

FIG. 6: Normal Liver, showing the hepatic lobule (left panel), hepatocytes (middle panel), and portal triad (right panel). Zone 1 Hepatocytes: moderate albumin expression (6-20 dots/cell); Zone 2 Hepatocytes: high albumin expression (21-50 dots/cell); and Zone 3 Hepatocytes: moderate albumin expression (6-20 dots/cell).

FIG. 7: Albumin expression in a sample of tissue from a Hepatocellular Carcinoma detected using RNA ISH.

FIG. 8: Albumin expression in a sample of tissue from an Intrahepatic Cholangiocarcinoma (IHCC) with high albumin expression, detected using RNA ISH.

FIG. 9: Albumin expression in a sample of tissue from an Intrahepatic Cholangiocarcinoma (IHCC) with low albumin expression, detected using RNA ISH.

FIG. 10: Albumin expression in a sample of tissue from Metastatic Liver Disease, detected using RNA ISH.

FIG. 11: An exemplary algorithm for detection of hepatic origin of metastatic tumors.

FIGS. 12A-D: A moderately differentiated hepatocellular carcinoma (A) with diffuse reactivity for Arginase-1 and focal reactivity for Hep Par 1 (B and C). The in situ hybridization stain for albumin is diffusely and strongly positive (D).

FIGS. 13A-D. A moderately differentiated hepatocellular carcinoma (A) negative for Arginase-1 and Hep Par 1, respectively (B and C). The in situ hybridization stain for albumin is diffusely and strongly positive (D).

FIGS. 14A-B. A clear cell variant of hepatocellular carcinoma (A) is diffusely and strongly positive for albumin (B). Note the dot-like pattern of reactivity.

FIGS. 15A-B: A poorly differentiated hepatocellular carcinoma (A) that was negative for Arginase-1 and Hep Par-1 (not shown) is positive for albumin (B). Note the dot-like pattern of reactivity.

### DETAILED DESCRIPTION

There is a significant unmet need for a sensitive and specific biomarker of primary liver tumors including BDA, IHCC and HCC. Because of the lack of a unique and reliable biomarker, these tumors are frequently classified as cancer of unknown origin or are labeled by pathologists as a tumor with pancreatico-biliary differentiation. Interestingly, expression profiling data supports the hypothesis that metastatic adenocarcinoma from the biliary tract is the single most common site of origin of a cancer of unknown primary (Hainsworth et al., Journal of Clinical Oncology 31:217-223, 2013; Yan et al., Am J Surg Pathol 34:1147-1154, 2010). This clinical conundrum may be resolved by albumin ISH where IHCCs were nearly all positive for albumin. Significantly, tumors that typically metastasize to the liver (gastrointestinal, pulmonary or breast adenocarcinomas) were negative for albumin. These neoplasms represent the closest histopathologic mimics of IHCC. However, a sensitive assay (such as a branched DNA (bDNA) platform used in the present Examples) is preferably used to detect and identify albumin mRNA, since in a substantial number of cases the expression of albumin was significantly less than that seen in hepatocellular carcinomas. The availability of such an assay would limit the need for extensive radiologic and endoscopic evaluations and help to reach treatment decisions in a timely fashion (Khan et al., Gut 61:1657-1669, 2012; Xiao et al., Histopathology 42:141-149, 2003). Furthermore, since this study was performed on tissue microarrays, these results are applicable to surgically resected tissue as well as more limited tissue samples such as fine needle biopsies. It should be noted that immunohistochemical assays for albumin are extremely difficult to interpret because of the ubiquitous presence of albumin in serum as well as the fact that both normal and neoplastic cells absorb albumin.

In addition, as described below, 22% of a cohort of intrahepatic carcinomas of uncertain origin were positive for albumin and hence are considered to represent (and reclassified as) IHCC. This data suggest that IHCC may be underrecognized, because of the inability to distinguish them from metastatic adenocarcinomas.

Albumin represents a highly specific and sensitive marker for the two most common primary tumors of the liver: hepatocellular carcinoma and cholangiocarcinoma. While pathologists typically cannot distinguish an IHCC from metastatic adenocarcinoma, the distinction between hepatocellular carcinoma and IHCC is generally straightforward: conventional histology and immunohistochemical techniques can readily distinguish the two neoplasms. In this series IHCCs were distinguished with certainty from hepatocellular carcinomas based on a H&E stain. In cases with overlapping features, the combination of glypican 3, Hep Par 1 and Arginase-1 has been shown to be a robust means of making this distinction (Yan et al., Am J Surg Pathol 34:1147-1154, 2010; Zhou et al., Oncogene. Nature 16:425-438, 2009).

Hepatic progenitor cells, a source of both hepatocytes and bile duct cells, express markers of both biliary epithelial cells (keratin 7, 19, 14) and hepatocytes (keratin 8, keratin 18, met, albumin; see Xiao et al., Histopathology 42:141-149, 2003; O'Dell et al., Cancer Research 72:1557-1567, 2012). While the transcript is silenced in the mature bile duct epithelium, the neo-ductules that emerge from regenerating liver express albumin. The presence of albumin thus supports the hypothesis that hepatocellular carcinoma and IHCC arise from a common progenitor cell. Genetically engineered models give further credence to this hypothesis where an albumin-Cre system with liver specific inactivation of Nf2, Mst1/Mst2 develop hepatocellular carcinoma and IHCC, although the predominant tumor type is hepatocellular carcinoma (Zhou et al., Oncogene. Nature 16:425-438, 2009; Kipp et al. Hum Pathol 43:1552-1558, 2012; Wang et al., Oncogene 32:3091-3100, 2012; Voss et al., Hum Pathol 44:1216-1222, 2013). A more recent animal model of IHCC that closely resembles its human counterpart used a albumin-Cre mediated somatic activation of Kras^{G12D} and deletion of Trp53: on histopathology, although the majority of tumors were IHCC, hepatocellular carcinomas were also identified (O'Dell et al., Cancer Research 72:1557-1567, 2012).

The perihilar as well as mid and distal bile duct carcinomas were uniformly negative for albumin. Although perihilar and distal bile duct carcinomas are also sometimes referred to as cholangiocarcinoma, recent data suggests that these tumors are genetically distinct from IHCC: e.g. mutations in IDH1/2 are much more common in IHCC (Kipp et al. Hum Pathol 43:1552-1558, 2012; Wang et al., Oncogene 32:3091-3100, 2012; Voss et al., Hum Pathol 44:1216-1222, 2013). Consequently, the clinical behavior of IHCC is distinct from distal bile duct carcinoma that has therapeutic implications including the prospect of IDH-targeted therapies that have shown promise in preclinical models of leukemia and glioma (Blechacz et al., Nat Rev Gastroenterol Hepatol 8:512-522, 2011; Razumilava et al., Clin Gastroenterol Hepatol 11:13-21.e1-quiz e3-4, 2013; Wang et al., Science 340:622-626, 2013; Rohle et al., Science 340:626-630, 2013). In addition to IDH1/2 mutations, genomic studies have also revealed actionable recurrent translocation events involving the FGFR2 locus in IHCCs (Borad et al., PLoS Genet. 2014; 10:e1004135).

The data presented herein also supports the hypothesis that IHCCs are biologically and genetically distinct from extrahepatic bile duct carcinomas; IHCC are derived from a progenitor cell capable of hepatic and cholangiocytic differentiation while the progenitor cell of bile duct carcinoma is restricted to cholangiocytic lineage. The current paradigm of making this distinction based on the epicenter of the lesion may prove inadequate thus this paradigm may be replaced by detecting albumin mRNA, e.g., using ISH. The molecular differences inherent to the cell of origin has implications for biological behavior, and therefore, represents a superior method to distinguish these cholangiocarcinomas for understanding response to therapy as well as designing cohorts for clinical trials.

Methods of Identifying Hepatic Neoplasms

The identity of malignant tumors remains equivocal in a significant number of patients and approximately 3% to 5% of all cancers have no identifiable primary site (Everhart and Ruhl, Gastroenterology 136:1134-1144, 2009; Muir, Cancer. 1; 75(1 Suppl):353-6, 1995), and IHCCs frequently fall into this category. The site of a tumor as well as the tumor type influence the success of molecular targeted therapies as the cellular context is as relevant as the mutational profile. For instance, the targeted RAF inhibitor, vemurafenib, is effective against melanomas with activated BRAF (BRAFV600E), but not against colorectal cancers harboring the same mutation (Chapman et al., N Engl J Med 364:2507-2516, 2011). Therefore, precise determination of primary tumor type as well as the site of origin remains a key diagnostic element to optimal treatment selection. The inability of histology and immunohistochemistry to determine tumor type in a minority of cases has spurred the development of gene expression signatures for tumor classification (Varadhachary et al., Journal of Clinical Oncology 26:4442-4448, 2008). However, these previous gene expression assays, such as the quantitative reverse transcription polymerase chain reaction (RT-PCR) assay used by Varadhachary et al., do not preserve morphological details, and hence the gene signature is an aggregate signal of tumor cells, stroma and other mesenchymal elements that accompany the tumor, thus decreasing their sensitivity and specificity. The measurement of albumin expression highlights the limitations of these gene expression assays as they would not be able to distinguish between albumin produced by the adjacent liver and tumor cells. Moreover, many of the informative genes recently used in determining site of origin are transcription factors (Hainsworth et al., Journal of Clinical Oncology 31:217-223, 2013), a class of proteins that is difficult to assess by conventional IHC but that would be amenable to an RNA-ISH assay. The RNA-ISH assay is also amenable to automation, and hence could be adopted by most diagnostic clinical laboratories, while most expression profiling assays require a send out to a centralized laboratory. The methods described herein that detect RNA in situ, e.g., in formalin fixed paraffin embedded material, fresh frozen tissue sections, fine needle aspirate biopsies, tissue microarrays, cells isolated from blood (including whole blood), bone marrow or sputum (such as samples prepared using centrifugation (such as with the CytoSpin Cytocentrifuge instrument (ThermoFisher Scientific, Waltham, Mass.) or smeared on a slide), blood smears on slides (including whole blood smears), and other sample types where the cellular morphology is sufficiently intact to allow the identification of the cells of interest (e.g., the cells that are albumin mRNA positive and negative), enable physicians to refine their diagnostic precision as well as provide novel prognostic and predictive biomarkers.

As described herein, normal hepatocytes and HCC show abundant expression of albumin mRNA. Bile duct epithelial cells and IHCC show a range of albumin expression, with some IHCC expressing abundant albumin whereas others show focal areas with low levels of albumin expression. BDA show low levels of albumin expression. Metastatic tumors in the liver show complete lack of albumin mRNA staining. Thus, Albumin ISH provides a sensitive and specific marker for primary liver tumors, e.g., IHCC. This test, which can be performed on, e.g., a needle biopsy, enables patients to forego numerous invasive and diagnostic tests, all of which cost time and money. Albumin ISH is a sensitive and specific test for the diagnosis of primary liver tumors such as IHCC and can be utilized, e.g., in patients with liver masses for whom a primary malignancy is not identified.

While hepatocytes strongly express albumin, the expression level of albumin in cholangiocytes is significantly lower than that in hepatocytes. Prior ISH studies lacked the desired sensitivity to detect low levels of albumin expression and have, therefore, not been successful in differentiating primary liver tumors, especially IHCC, from metastatic liver disease.

Low levels of albumin expression, especially in cholangiocytes, can be detected using RNA ISH with bDNA signal amplification technology, to detect albumin expression in tumors of both, hepatocyte and cholangiocyte origins. Additionally, using high specificity RNA ISH bDNA detection methods, albumin expression can be detected only in HCC, IHCC, and BDA, and not in metastatic liver disease. It is important to distinguish primary malignant liver neoplasms (HCC & IHCC), as well as benign liver tumors such as BDA, from metastatic liver disease, and to distinguish HCC and IHCC from BDA; the distinction between the two types of primary malignant liver neoplasms (i.e., between HCC and IHCC) can be made by routine light microscopy using H/E staining.

Histologically, the liver is divided into lobules, at the center of which is the central vein. At the periphery of the lobule are the portal triads. Functionally, the liver can be divided into acini with three zones, based upon oxygen supply. Zone 1 surrounds the portal tracts where the oxygenated hepatic arterial and portal venous blood enters. Zone 3 is located around the central veins where oxygenation is poor. Zone 2 is located in between Zones 1 and 3.

Normal hepatocytes can be distinguished from tumor cells by dissimilar staining patterns. For example, in an RNA ISH assay where the signal is generated by the use of fast red with an alkaline phosphatase, normal hepatocytes have yellowish-orange cytoplasm with dark-red ISH dots whereas tumor cells have clear cytoplasm with dark red ISH dots.

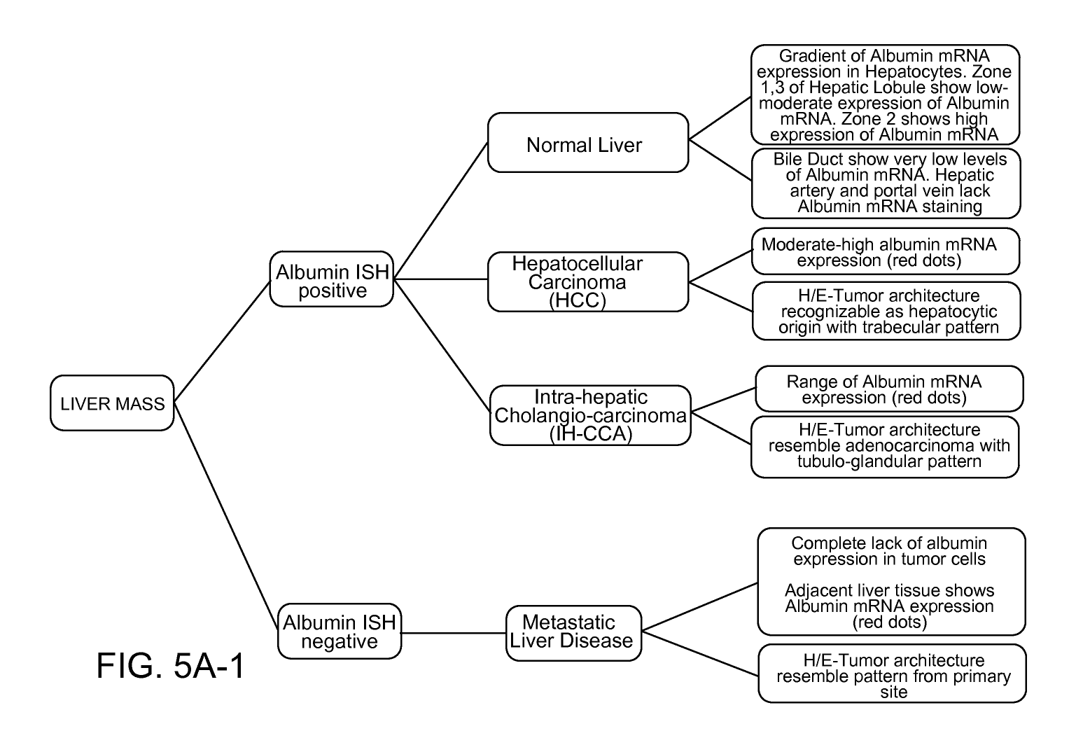

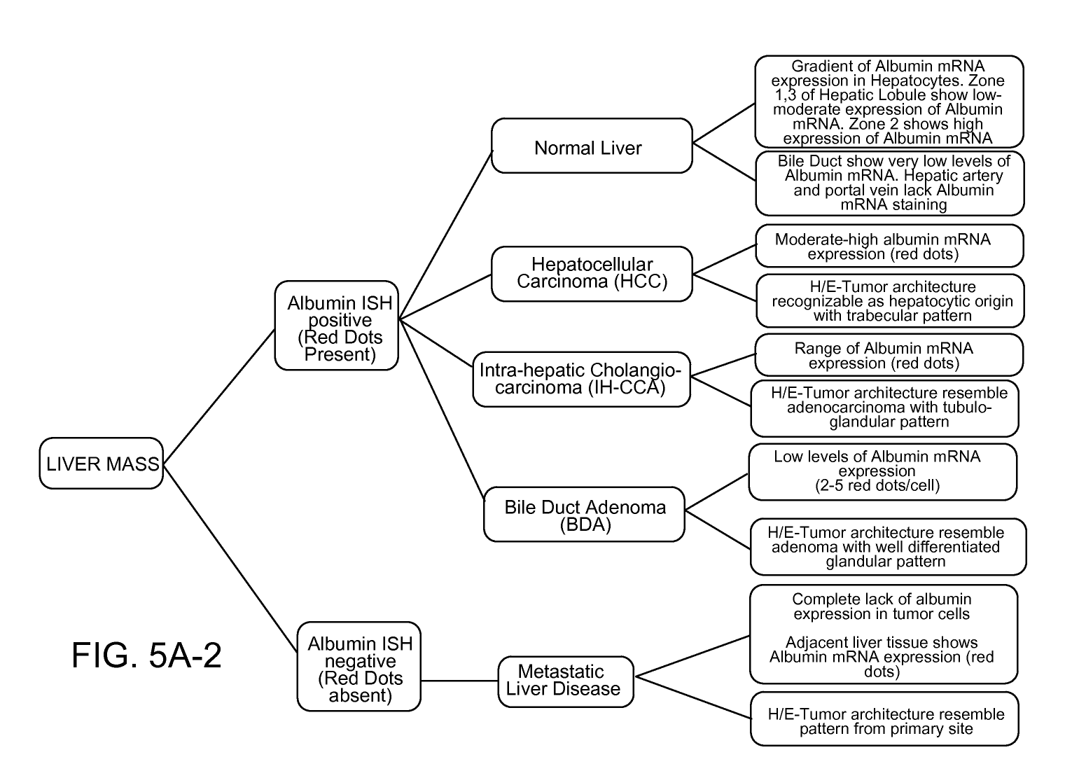

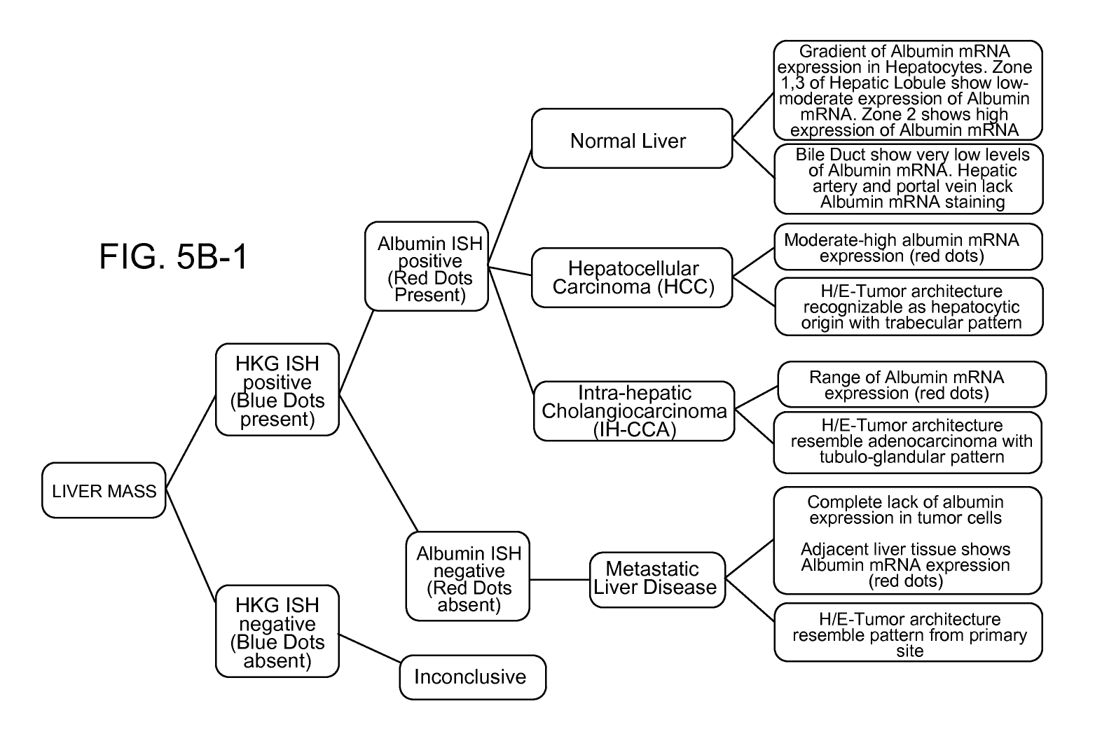

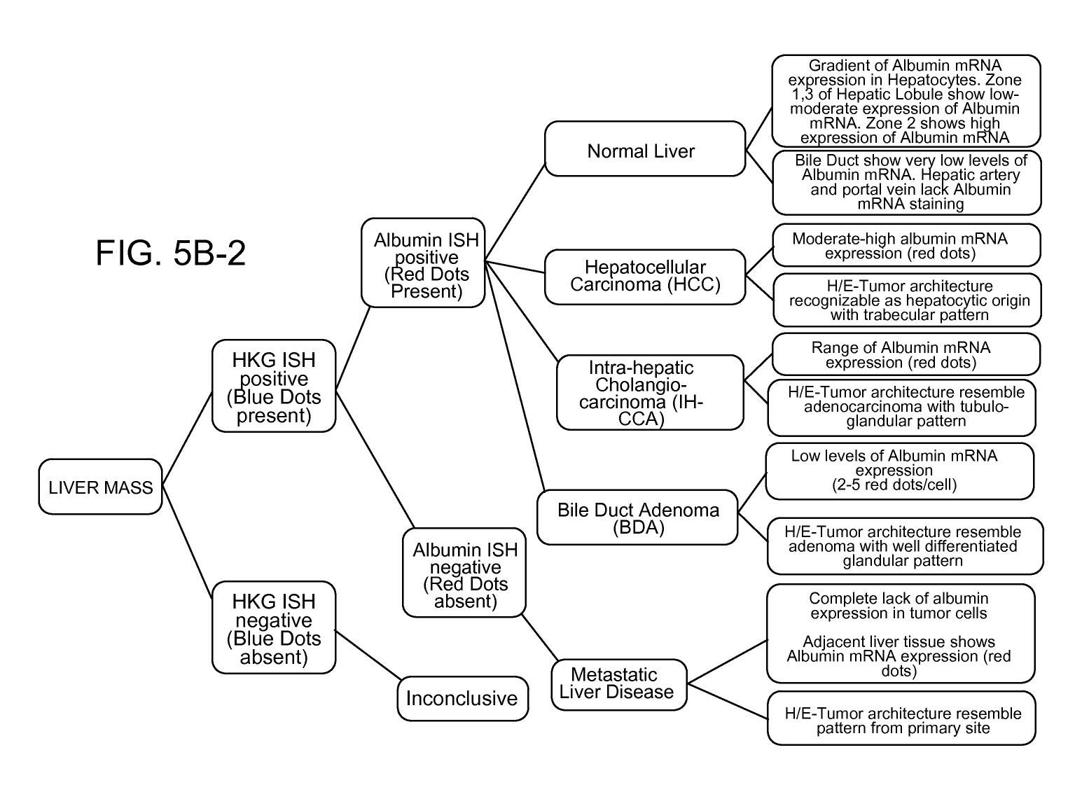

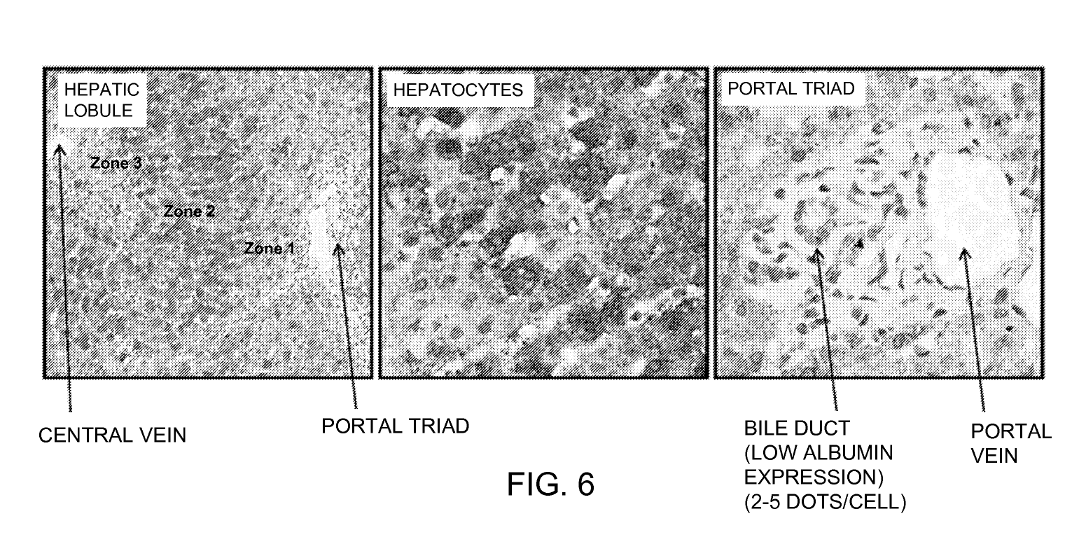

FIGS. 5A1-2 and 5B1-2 outline exemplary algorithms for the differential diagnosis of HCC/IHCC/BDA from metastatic liver disease. The following factors can be used to make the differential diagnosis:
A. Normal Liver has the following morphological features seen by ISH (see, e.g., FIG. 6):
   1. Sheets of hepatocytes showing gradient of albumin ISH staining in the Hepatic Lobule.
      a. Zone 1 (Peri-portal) showing moderate levels (6-20 dots) of albumin mRNA.
      b. Zone 2 (Intermediate) showing high levels of albumin mRNA (21-50 dots).
      c. Zone 3 (Centri-lobular) showing moderate levels of albumin mRNA (6-20 dots).
   2. Portal triad showing bile duct with low-levels of albumin mRNA (2-5 dots). Hepatic artery and portal vein show complete lack of albumin mRNA staining
B. Deciduous normal hepatocytes can be distinguished from tumor cells by the distinctive staining pattern observed after ISH. Deciduous hepatocytes show yellowish-orange cytoplasm as compared to tumor cells which exhibit clear cytoplasm. In addition, deciduous normal hepatocytes show moderate levels of albumin expression (6-20 dots) as compared to tumor cells which show variable levels of albumin expression.
C. HCC has the following morphological features seen by ISH:
   1. Tumor cells showing moderate-high levels (6-50 dots) of albumin mRNA staining.
   2. H/E staining shows trabecular arrangement of tumor cells resembling normal hepatocytes.
D. IHCC has the following morphological features seen by ISH:
   1. Tumor cells showing variable levels of albumin mRNA staining. Some tumors may show high levels of albumin mRNA staining while some tumors may show moderate or low levels of albumin mRNA staining. The tumor may show generalized expression of albumin or may show focal areas of albumin expression.
   2. H/E staining shows tubulo-glandular arrangement of tumor cells resembling adenocarcinoma.
E. Bile Duct Adenoma (BDA) has the following morphological features seen by ISH:
   1. Tumor cells showing low levels of Albumin mRNA staining (2-5 red dots/cell).
   2. H/E staining shows well differentiated glandular arrangement of tumor cells resembling adenoma.
F. Metastatic Liver Disease has the following morphological features seen by ISH:
   1. Tumor cells showing complete lack of albumin mRNA staining. Surrounding hepatocytes show moderate-high levels (6-50 dots) of albumin mRNA staining.
   2. H/E staining shows tumor architecture resembling pattern from the primary site.
G. For all cases the following should be excluded from consideration:
   1. Staining outside of the cytoplasm of cell
   2. Cells showing presence of nuclear staining on ISH

Methods of Identifying Metastases of Hepatic Origin

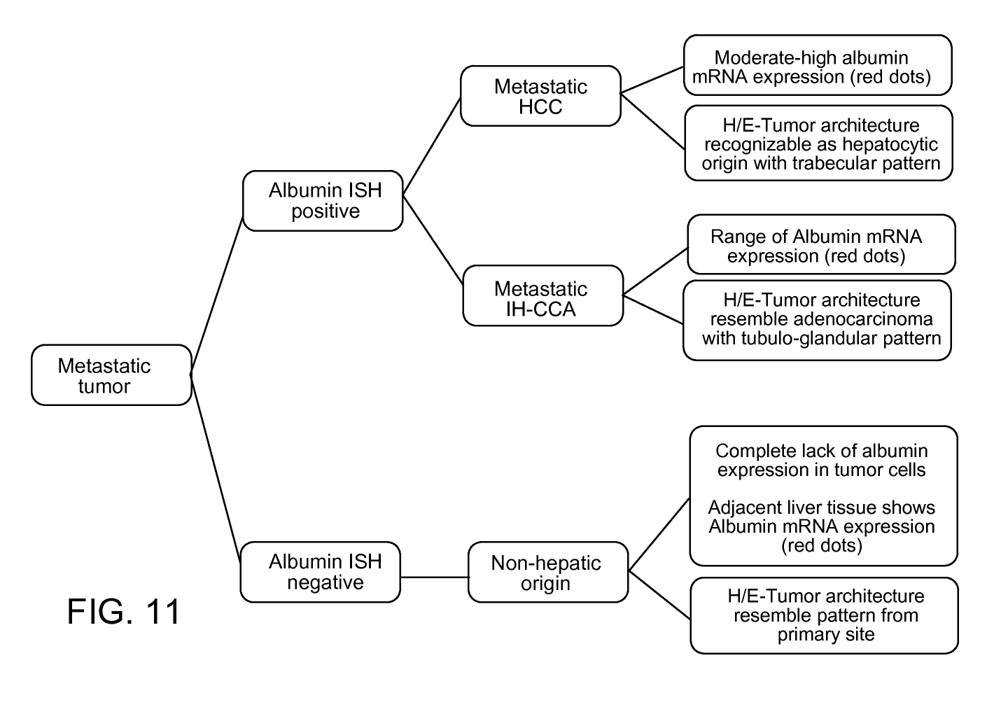

Since albumin is synthesized exclusively by hepatocytes, the present methods, preferably using an albumin RNA ISH assay, can be used for the detection of metastatic lesions derived from hepatic origin. In tumors originating from the liver, the metastatic tumor cells show positive staining for albumin mRNA surrounded by cells lacking albumin mRNA expression (see FIG. 11 for an exemplary algorithm for detection of hepatic origin of metastatic tumors). Tumors originating from tissues other than the liver would be negative for albumin mRNA.

Methods for Detection of Albumin mRNA

The methods described herein include the detection and optionally quantitation of albumin mRNA in situ in cell and tissue samples, e.g., in formalin fixed paraffin embedded (FFPE) biopsy or tissue samples, fresh frozen tissue sections, fine needle aspirate biopsies, tissue microarrays, cells isolated from blood (including whole blood), bone marrow or sputum (such as samples prepared using centrifugation (such as with the CytoSpin Cytocentrifuge (ThermoFisher Scientific, Waltham, Mass.) or smeared on a slide), blood smears on slides (including whole blood smears), and other sample types where the cellular morphology is sufficiently intact to allow the identification of the cells of interest (e.g., the cells that are albumin mRNA positive and negative). Preferably the samples are tissue samples with low tumor cellularity (i.e., a low proportion of tumor cells relative to normal cells in a sample).

This detection can be performed using methods known in the art; a preferred method is RNA in situ hybridization (RNA ISH). Other methods known in the art for gene expression analysis, e.g., RT-PCR, RNA-sequencing, and oligo hybridization assays including RNA expression microarrays, hybridization based digital barcode quantification assays such as the nCounter® System (NanoString Technologies, Inc., Seattle, Wash.), and lysate based hybridization assays utilizing branched DNA signal amplification such as the QuantiGene 2.0 Single Plex and Multiplex Assays (Affymetrix, Inc., Santa Clara, Calif.); however, these non-RNA ISH methods cannot visualize RNA in situ, which is important in identifying the cell of origin. Visualizing RNA in situ is often important in distinguishing cells that may have varying levels of expression for a particular RNA, identifying heterogeneity within tumors, distinguishing between tumor and non-tumor cells, and so on. Thus in some embodiments of the methods described herein RNA ISH methods are used wherein the cells are individually identifiable (i.e., although the cells are permeabilized to allow for influx and outflux of detection reagents, the structure of individual cells is maintained such that each cell can be identified); in contrast, methods such as RT-PCR, expression arrays, and so on use bulk samples wherein the RNA is extracted from disrupted cells, and the cells are not identifiable (and thus the cell of origin cannot be identified). RNA ISH methods also provide a distinct advantage when used to analyze RNAs for which the corresponding proteins are secreted as an immunohistochemistry approach for such biomarkers would simply show the presence of protein throughout the tissue at issue, even if certain cells are not expressing or are expressing at a different level than the average expression within the assayed sample.

Certain RNA ISH platforms leverage the ability to amplify the signal within the assay via a branched-chain technique of multiple polynucleotides hybridized to one another (e.g., bDNA) to form a branch structure (e.g., branched nucleic acid signal amplification). In addition to its high sensitivity, the platform also has minimal non-specific background signal compared to immunohistochemistry. While RNA ISH has been used in the research laboratory for many decades, tissue based RNA diagnostics have only recently been introduced in the diagnostic laboratory. However, these have been restricted to highly expressed transcripts such as immunoglobulin light chains as low abundance transcripts otherwise cannot be detected by a conventional RNA ISH platform (Hong et al., Surgery 146:250-257, 2009; Magro et al., J Cutan Pathol 30:504-511, 2003). This robust RNA ISH platform with its ability to detect low transcript numbers has the potential to revolutionize RNA diagnostics in paraffin tissue and other tissue assay sample formats. The ability in this study to detect albumin in virtually all IHCCs with minimal non-specific signal is a significant advance when compared to prior studies that have failed to detect albumin in this neoplasm reliably (Tickoo et al., Am J Surg Pathol 26:989, 2002; Krishna et al., Am J Surg Pathol 21:147-152, 1997; Murray et al., J Clin Pathol 45:21-24, 1992).

In some embodiments, the assay is a bDNA assay. Optionally, the assay is a bDNA assay as described in U.S. Pat. Nos. 7,709,198; 7,803,541; 8,114,681 and 2006/0263769, which describe the general bDNA approach; see especially 14:39 through 15:19 of the '198 patent. In some embodiments, the methods include using a modified RNA in situ hybridization (ISH) technique using a branched-chain DNA assay to directly detect and evaluate the level of biomarker mRNA in the sample (see, e.g., Luo et al., U.S. Pat. No. 7,803,541B2, 2010; Canales et al., Nature Biotechnology 24(9):1115-1122 (2006); Ting et al., Aberrant Overexpression of Satellite Repeats in Pancreatic and Other Epithelial Cancers, Science 331(6017):593-6 (2011)). A kit for performing this assay is commercially-available from Affymetrix, Inc. (e.g., the ViewRNA™ Assays for tissue and cell samples within both manual and automated formats).

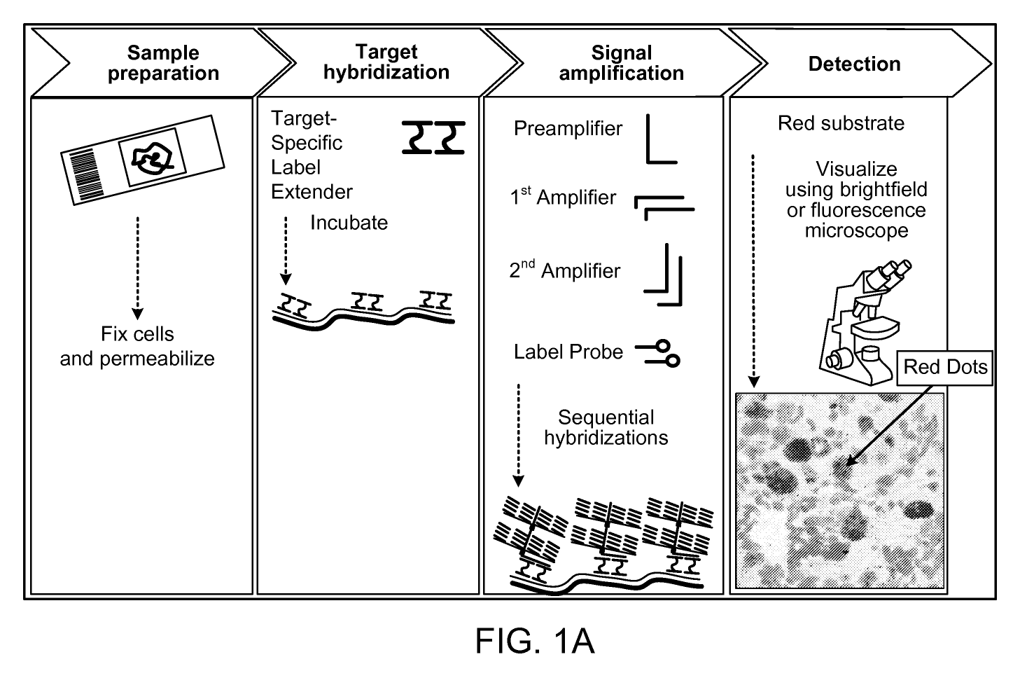

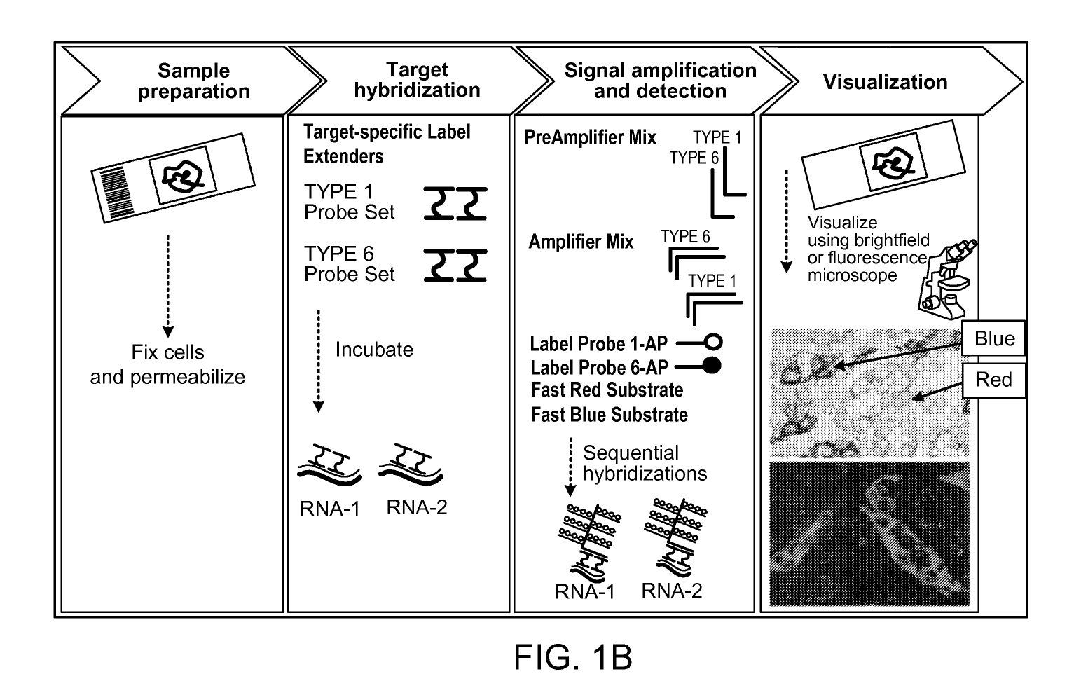

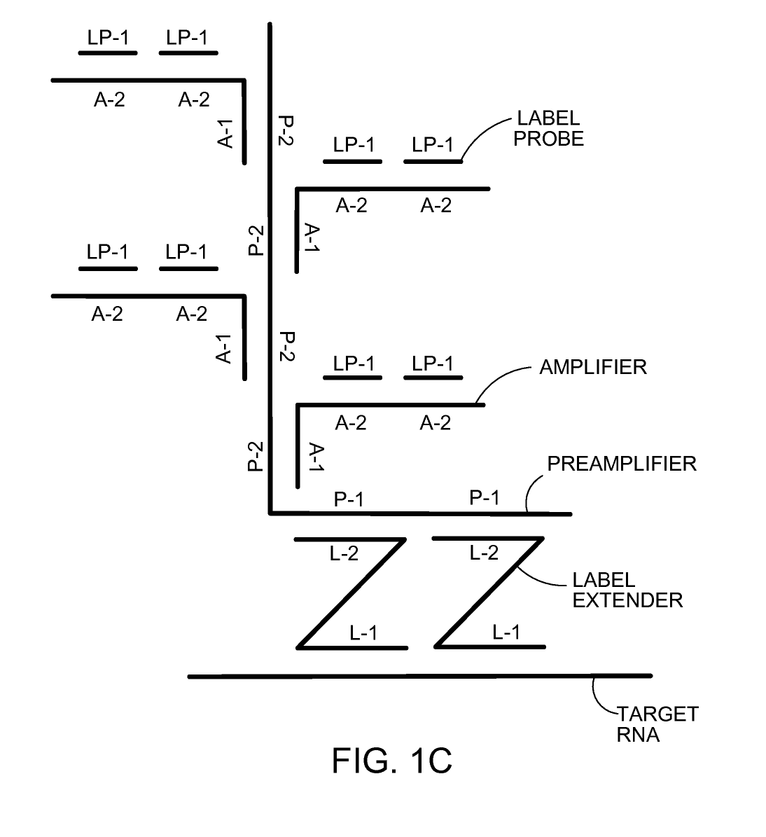

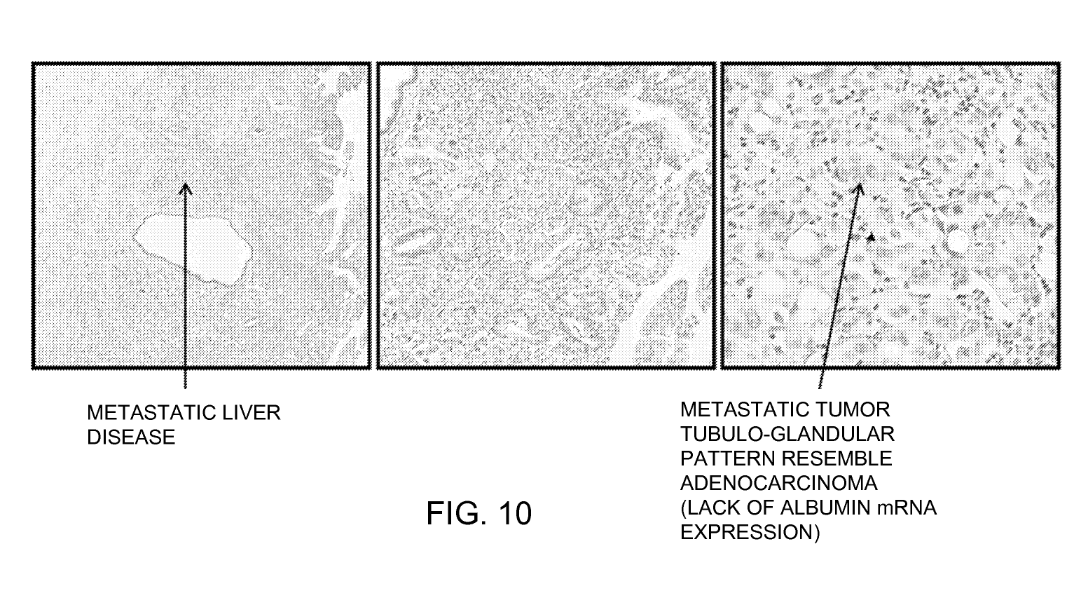

RNA ISH can be performed, e.g., using the ViewRNA™ technology (Affymetrix, Santa Clara, Calif.). ViewRNA™ ISH is based on the branched DNA technology wherein signal amplification is achieved via a series of sequential steps (e.g., as shown in FIG. 1A in a single plex format and in FIG. 1B in a two plex format). Thus in some embodiments, the methods include performing an assay as described in US 2012/0052498 (which describes methods for detecting both a nucleic acid and a protein with bDNA signal amplification, comprising providing a sample comprising or suspected of comprising a target nucleic acid and a target protein; incubating at least two label extender probes each comprising a different L-1 sequence, an antibody specific for the target protein, and at least two label probe systems with the sample comprising or suspected of comprising the target nucleic acid and the target protein, wherein the antibody comprises a pre-amplifier probe, and wherein the at least two label probe systems each comprise a detectably different label; and detecting the detectably different labels in the sample); US 2012/0004132; US 2012/0003648 (which describes methods of amplifying a nucleic acid detection signal comprising hybridizing one or more label extender probes to a target nucleic acid; hybridizing a pre-amplifier to the one or more label extender probes; hybridizing one or more amplifiers to the pre-amplifier; hybridizing one or more label spoke probes to the one or more amplifiers; and hybridizing one or more label probes to the one or more label spoke probes); or US 2012/0172246 (which describes methods of detecting a target nucleic acid sequence, comprising providing a sample comprising or suspected of comprising a target nucleic acid sequence; incubating at least two label extender probes each comprising a different L-1 sequence, and a label probe system with the sample comprising or suspected of comprising the target nucleic acid sequence; and detecting whether the label probe system is associated with the sample). Each hybridized target specific polynucleotide probe acts in turn as a hybridization target for a pre-amplifier polynucleotide that in turn hybridizes with one or more amplifier polynucleotides. In some embodiments two or more target specific probes (label extenders) are hybridized to the target before the appropriate pre-amplifier polynucleotide is bound to the 2 label extenders, but in other embodiments a single label extender can also be used with a pre-amplifier. Thus, in some embodiments the methods include incubating one or more label extender probes with the sample. In some embodiments, the target specific probes (label extenders) are in a ZZ orientation, cruciform orientation, or other (e.g., mixed) orientation; see, e.g., FIGS. 10A and 10B of US 2012/0052498. Each amplifier molecule provides binding sites to multiple detectable label probe oligonucleotides, e.g., alkaline phosphatase (AP)-conjugated-polynucleotides, thereby creating a fully assembled signal amplification “tree” that has numerous binding sites for the label probe; the number of binding sites can vary depending on the tree structure and the labeling approach being used, e.g., from 16-64 binding sites up to 3000-4000 range. In some embodiments there are 300-5000 probe binding sites. The number of binding sites can be optimized to be large enough to provide a strong signal but small enough to avoid issues associated with overlarge structures, i.e., small enough to avoid steric effects and to fairly easily enter the fixed/permeabilized cells and be washed out of them if the target is not present, as larger trees will require larger components that may get stuck within pores of the cells (e.g., the pores created during permeabilization, the pores of the nucleus) despite subsequent washing steps and lead to noise generation. A simplified exemplary bDNA amplification scheme is shown in FIG. 1C, the components of which can be modified in a variety of means, including as described herein and within the preceding references.

Where an alkaline phosphatase (AP)-conjugated polynucleotide probe is used, following sequential addition of an appropriate substrate such as fast red or fast blue substrate, AP breaks down the substrate to form a precipitate that allows in-situ detection of the specific target RNA molecule. Alkaline phosphatase can be used with a number of substrates, e.g., fast red, fast blue, or 5-Bromo-4-chloro-3-indolyl-phosphate (BCIP). Thus in some embodiments, the methods include the use of alkaline phosphatase conjugated polynucleotide probes within a bDNA signal amplification approach, e.g., as described generally in U.S. Pat. No. 5,780,277 and U.S. Pat. No. 7,033,758.

Other embodiments include the use of fluorophore-conjugates probes, e.g., Alexa Fluor conjugated label probes, or utilize other enzymatic approaches besides alkaline phosphatase for a chromogenic detection route, such as the use of horseradish peroxidase conjugated probes with substrates like 3,3′-Diaminobenzidine (DAB).

In some embodiments, the assay is similar to those described in US 2012/0100540; US 2013/0023433; US 2013/0171621; US 2012/0071343; or US 2012/0214152. All of the foregoing are incorporated herein by reference in their entirety.

The assay can be conducted manually or on an automated instrument, such the Leica BOND family of instruments, or the Ventana DISCOVERY ULTRA or DISCOVERY XT instruments.

In some embodiments, the detection methods use a 1-plex format with an RNA probe set targeting the human albumin mRNA transcripts, e.g., as shown in FIG. 1A. The presence of albumin signals in the sample of tumor tissue, or the absence thereof, allows the differential diagnosis of HCC-CCA (when albumin mRNA is present, or present above a threshold level) from metastatic liver disease (when albumin mRNA is absent, or present below a threshold level); an exemplary decision tree is shown in FIG. 5A-1. In some embodiments, the detection methods use a 2-plex format in combination with probe sets targeting one or more pan-housekeeping (pan-HKG) genes, e.g. GAPDH, ACTB, or UBC, to assess RNA integrity, e.g., as shown in FIG. 1B. Cells that do not have expression of pan-HKG lack essential RNA integrity and hence need to be excluded from the analysis; exemplary decision trees are shown in FIGS. 5B-1 and 5B-2. This eliminates false negative cases.

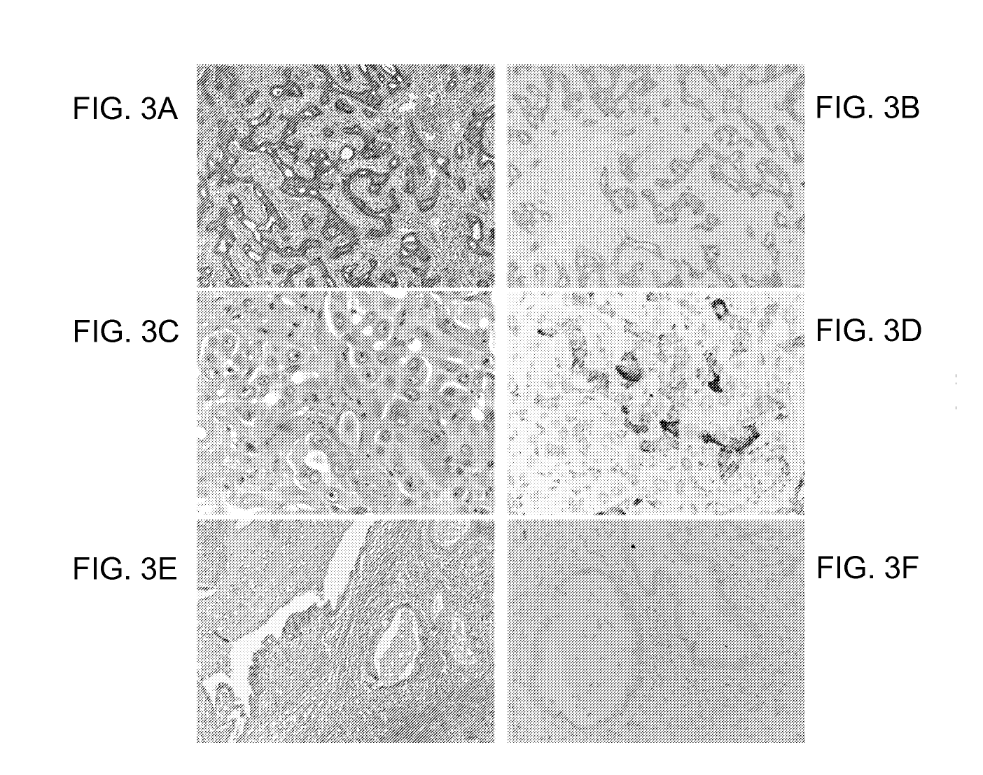

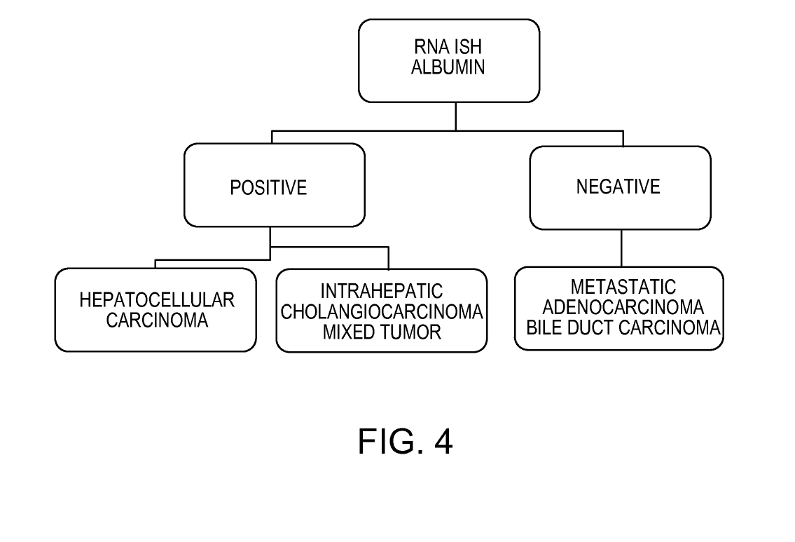

The sequence of human albumin is known in the art, see, e.g., FIG. 3 of Dugaiczyk et al., Proc Natl Acad Sci USA. 79(1): 71-75 (1982); the mRNA sequence is shown below, and is available in GenBank under Acc. No. NM_{—}000477.5, and the protein sequence is available in GenBank under Acc. No. NP_{—}000468.1. Other species are known in the art and include: M. musculus, Acc. No. NP_{—}033784.2; Canis lupus familiaris, Acc. No. NM_{—}001003026.1; Equus caballus, Acc. No. NP_{—}001075972.1; Felis domesticus, Acc. No. X84842. One of skill in the art would readily be able to identify sequences for additional species bioinformatically, and would appreciate that the sequence of albumin mRNA used should match the species of the subject from which the sample is obtained. The subject is preferably a mammal and can be, e.g., a human or veterinary subject (e.g., cat, dog, horse, cow, or sheep).

Human Albumin
(SEQ ID NO: 1)

| | |
|---|---|
| 1 | agtatattag tgctaatttc cctccgtttg tcctagcttt |
| | tctcttctgt caaccccaca |
| 61 | cgcctttggc acaatgaagt gggtaacctt tatttccctt |
| | ctttttctct ttagctcggc |
| 121 | ttattccagg ggtgtgtttc gtcgagatgc acacaagagt |
| | gaggttgctc atcggtttaa |
| 181 | agatttggga gaagaaaatt tcaaagcctt ggtgttgatt |
| | gcctttgctc agtatcttca |
| 241 | gcagtgtcca tttgaagatc atgtaaaatt agtgaatgaa |
| | gtaactgaat ttgcaaaaac |
| 301 | atgtgttgct gatgagtcag ctgaaaattg tgacaaatca |
| | cttcataccc tttttggaga |
| 361 | caaattatgc acagttgcaa ctcttcgtga aacctatggt |
| | gaaatggctg actgctgtgc |
| 421 | aaaacaagaa cctgagagaa atgaatgctt cttgcaacac |
| | aaagatgaca acccaaacct |
| 481 | cccccgattg gtgagaccag aggttgatgt gatgtgcact |
| | gcttttcatg acaatgaaga |
| 541 | gacatttttg aaaaaatact tatatgaaat tgccagaaga |
| | catccttact tttatgcccc |
| 601 | ggaactcctt ttctttgcta aaaggtataa agctgctttt |
| | acagaatgtt gccaagctgc |
| 661 | tgataaagct gcctgcctgt tgccaaagct cgatgaactt |
| | cgggatgaag ggaaggcttc |
| 721 | gtctgccaaa cagagactca agtgtgccag tctccaaaaa |
| | tttggagaaa gagctttcaa |
| 781 | agcatgggca gtagctcgcc tgagccagag atttcccaaa |
| | gctgagtttg cagaagtttc |
| 841 | caagttagtg acagatctta ccaaagtcca cacggaatgc |
| | tgccatggag atctgcttga |
| 901 | atgtgctgat gacagggcgg accttgccaa gtatatctgt |
| | gaaaatcaag attcgatctc |
| 961 | cagtaaactg aaggaatgct gtgaaaaacc tctgttggaa |
| | aaatcccact gcattgccga |
| 1021 | agtggaaaat gatgagatgc ctgctgactt gccttcatta |
| | gctgctgatt ttgttgaaag |
| 1081 | taaggatgtt tgcaaaaact atgctgaggc aaaggatgtc |
| | ttcctgggca tgtttttgta |
| 1141 | tgaatatgca agaaggcatc ctgattactc tgtcgtgctg |
| | ctgctgagac ttgccaagac |
| 1201 | atatgaaacc actctagaga agtgctgtgc cgctgcagat |
| | cctcatgaat gctatgccaa |
| 1261 | agtgttcgat gaatttaaac ctcttgtgga agagcctcag |
| | aatttaatca aacaaaattg |
| 1321 | tgagcttttt gagcagcttg gagagtacaa attccagaat |
| | gcgctattag ttcgttacac |
| 1381 | caagaaagta ccccaagtgt caactccaac tcttgtagag |
| | gtctcaagaa acctaggaaa |
| 1441 | agtgggcagc aaatgttgta aacatcctga agcaaaaaga |
| | atgccctgtg cagaagacta |
| 1501 | tctatccgtg gtcctgaacc agttatgtgt gttgcatgag |
| | aaaacgccag taagtgacag |
| 1561 | agtcaccaaa tgctgcacag aatccttggt gaacaggcga |
| | ccatgctttt cagctctgga |
| 1621 | agtcgatgaa acatacgttc ccaaagagtt taatgctgaa |
| | acattcacct tccatgcaga |
| 1681 | tatatgcaca ctttctgaga aggagagaca aatcaagaaa |
| | caaactgcac ttgttgagct |
| 1741 | cgtgaaacac aagcccaagg caacaaaaga gcaactgaaa |
| | gctgttatgg atgatttcgc |
| 1801 | agcttttgta gagaagtgct gcaaggctga cgataaggag |
| | acctgctttg ccgaggaggg |
| 1861 | taaaaaactt gttgctgcaa gtcaagctgc cttaggctta |
| | taacatcaca tttaaaagca |
| 1921 | tctcagccta ccatgagaat aagagaaaga aaatgaagat |
| | caaaagctta ttcatctgtt |
| 1981 | tttctttttc gttggtgtaa agccaacacc ctgtctaaaa |
| | aacataaatt tctttaatca |
| 2041 | ttttgcctct tttctctgtg cttcaattaa taaaaaatgg |
| | aaagaatcta atagagtggt |
| 2101 | acagcactgt tatttttcaa agatgtgttg ctatcctgaa |
| | aattctgtag gttctgtgga |
| 2161 | agttccagtg ttctctctta ttccacttcg gtagaggatt |
| | tctagtttct tgtgggctaa |
| 2221 | ttaaataaat cattaatact cttctaaaaa aaaaaaaaaa |
| | aaaa |

Methods of Treatment

The methods described herein can also be used to treat a subject, or to guide selection of a treatment. For example, once a diagnosis has been made, a treatment can be selected and optionally administered to a subject. Treatments for the conditions described herein are known in the art.

Treatments for use in the methods described herein can include a surgical treatment and/or the administration of a therapeutic agent. Therapeutic agents include sorafenib and/or chemotherapeutic agents e.g. 5-FU, capecitabine, gemcitabine, cisplatin and/or oxaliplatin. Treatments for HCC include surgical (e.g., liver resection, liver transplantation), ablative (e.g., transarterial chemoembolization (TACE), radiofrequency ablation (RFA), radioembolisation (e.g., with ⁹⁰Y spheres) and/or chemical (e.g., sorafenib). Treatments for IHCC include surgical (e.g., liver resection, liver transplantation), ablative (e.g., transarterial chemoembolization (TACE), radiofrequency ablation (RFA), radioembolisation (e.g., with 90Y spheres), stenting, radiotherapy, and/or chemical (e.g., chemotherapy with 5-FU, capecitabine, gemcitabine, cisplatin, oxaliplatin).

Thus, for example, when a sample comprising tumor cells from a subject is identified as having HCC or IHCC or BDA, or metastatic disease, a treatment is identified, selected, and/or optionally administered to the subject. An exemplary treatment grid is shown in Table 2.

TABLE 2
Treatment Grid

| RNA-ISH and | | | |
|---|---|---|---|
| H&E | H&E | Diagnosis | Treatment |
| Positive - | Tumor | HCC | surgical (e.g., liver resection, |
| moderate-high | architecture | | liver transplantation), ablative |
| albumin mRNA | recognizable as | | (e.g., transarterial |
| expression | hepatocytic origin | | chemoembolization (TACE), |
| | with trabecular | | radiofrequency ablation (RFA), |
| | pattern | | radioembolisation (e.g., with |
| | | | ⁹⁰Y spheres) and/or chemical |
| | | | (e.g., sorafenib)^{A} |
| Positive - | Tumor | IHCC | surgical (e.g., liver resection, |
| Range of | architecture | | liver transplantation), ablative |
| Albumin mRNA | recognizable as | | (e.g., transarterial |
| expression | hepatocytic origin | | chemoembolization (TACE), |
| | with trabecular | | radiofrequency ablation (RFA), |
| | pattern | | radioembolisation (e.g., with |
| | | | 90Y spheres), stenting, |
| | | | radiotherapy, and/or chemical |
| | | | (e.g., chemotherapy with 5-FU, |
| | | | capecitabine, gemcitabine, |
| | | | cisplatin, oxaliplatin)^{B} |
| Positive - low | tumor architecture | BDA | conservative management, |
| levels of Albumin | resembling | | including but not limited to |
| mRNA | adenoma with a | | periodic monitoring or surgical |
| expression | well differentiated | | resection |
| | glandular pattern | | |
| Negative - | Tumor | Metastatic | Depends on identity of primary site^{C} |
| Complete lack of | architecture | Liver | |
| albumin | resemble pattern | Disease | |
| expression in | from primary site | | |
| tumor cells; | | | |
| Adjacent normal | | | |
| liver tissue shows | | | |
| albumin mRNA | | | |
| expression | | | |

^{A}Bruix and Sherman, Hepatology 53(3): 1020-1022 (2011); Jelic and Sotiropoulos, Ann Oncol 21(suppl 5): v59-v64 (2010); EASL-EORTC, Journal of Hepatology 56: 908-943 (2012).
^{B}Khan et al., Gut. 61(12): 1657-1669 (2012); Eckel et al., Ann Oncol 21(suppl 5): v65-v69 (2010); Khan Gut 51: vil-vi9 (2002); Hezel AF and Zhu AX, The Oncologist. (13): 415-423 (2008).
D: See, e.g., the NCCN cancer treatment guidelines; ASCO treatment guidelines; ESMO treatment guidelines; Oxford Textbook of Oncology, Second Edition; Textbook of Medical Oncology, Informa Healthcare; Comprehensive Textbook of Oncology.

### EXAMPLES

The invention is further described in the following examples, which do not limit the scope of the invention described in the claims.

Materials and Methods

The following Materials and Methods were used in the Examples below.

Study Cases

The study cases were selected based on a search of clinical and pathology databases. Formalin fixed pathologic specimens of primary liver adenocarcinomas, hepatocellular carcinoma, and normal liver cases were selected. The distinction of an IHCC from perihilar carcinomas was based on evaluation of the surgical specimen, imaging and the operative report.

Adenocarcinomas evaluated included tumors arising in the lung (n=22), esophagus and gastroesophageal junction (N=40), stomach (N=72), colon (N=40), pancreas (N=210), renal cell carcinoma (n=46), breast (n=34), bladder (N=8), endometrium (N=8), and ovary (N=6), representing the most common carcinomas that metastasize to the liver. In addition to known tumor types, 27 tumors of unknown origin were evaluated. All tumor types were placed on tissue microarrays constructed using 2×2 mm cores of paraffin embedded tissue. Entire sections of 30 IHCC and 30 pancreatic ductal adenocarcinomas were also evaluated. Staining with GAPDH/keratin was performed to validate the preservation of mRNA.

RNA ISH Platform

ViewRNA™ ISH is based on the branched DNA technology wherein signal amplification is achieved via a series of sequential steps. Each pair of bound target probe oligonucleotides acts as a template to hybridize a pre-amplifier molecule that in turn binds multiple amplifier molecules. Each amplifier molecule provides binding sites to multiple alkaline phosphatase (AP)-conjugated-oligonucleotides thereby creating a fully assembled signal amplification “tree” that has approximately 400 binding sites for the AP-labeled probe. Following sequential addition of the fast-red substrate, AP breaks down the substrate to form a precipitate (which forms a red dot that can be visualized by, e.g., a standard brightfield or fluorescent microscope) that allows in-situ detection of the specific target RNA molecule. The theoretical lower limit of detection is a single RNA transcript.

RNA ISH probe sets for the ViewRNA™ Assay (Affymetrix, Santa Clara, Calif.) were designed against albumin transcripts as identified in the NCBI nucleotide database (GenBank Acc. No. NM_{—}000477.5).

Briefly, dissected tissues were fixed for <24 hours in 10% Neutral Buffer Formalin at room temperature, followed by the standard formaldehyde-fixed, paraffin-embedded (FFPE) preparation. The FFPE tissues were sectioned at 5+/−1 micron and mounted on Surgipath X-tra® glass slides (Leica BioSystems, Buffalo Grove, Ill.), baked for 1 hour at 60° C. to ensure tissue attachment to the glass slides, and then subjected to xylene deparaffinization and ethanol dehydration. To unmask the RNA targets, dewaxed sections were incubated in 500 ml pretreatment buffer at 90-95° C. for 10 minutes and digested with 1:100 dilution protease at 40° C. for 10-20 minutes, followed by fixation with 10% formaldehyde at room temperature for 5 minutes. Unmasked tissue sections were subsequently hybridized with 1:40 dilution albumin probe sets for 2 hours at 40° C., followed by series of post-hybridization washes. Signal amplification was achieved via a series of sequential hybridizations and washes as described in the user's manual. Slides were post-fixed with 4% formaldehyde, counterstained with Gill's hematoxylin, mounted using Dako Ultramount (Dako, Carpinteria, Calif.), and visualized using a standard bright-field microscope.

Automated ISH assays for mRNA were performed using ViewRNA™ RX reagents (Affymetrix, Santa Clara, Calif.) on the BOND RX advanced staining system controlled with BDZ 5.0 software (Leica Biosystems, Buffalo grove, IL). Tissue sections on slides were processed automatically from deparaffinization, through ISH staining to hematoxylin counterstaining; sections were coverslipped off-instrument. Briefly, 5 micron sections of FFPE tissue were mounted on Surgipath X-tra glass slides, baked for 1 hour at 60° C. and placed on the BOND RX for processing. The BOND RX user-selectable settings were the ViewRNA 1 protocol and ViewRNA Dewax1; ViewRNA HIER 10 min, ER2 (90); ViewRNA Enzyme1 (20); ViewRNA Probe Hybridization. With these settings, the RNA unmasking conditions for the liver tissue consisted of a 10 min incubation at 90° C. in Bond Epitope Retrieval Solution 2 (Leica Biosystems, Buffalo grove, IL) followed by 10 min incubation with Proteinase K from the BOND Enzyme Pretreatment Kit at 1:1000 dilution (Leica Biosystems, Buffalo Grove, Ill.). Albumin and GAPDH mRNA-targeting Probe Sets were diluted 1:20 in ViewRNA Probe Diluent (Affymetrix, Santa Clara, Calif.) for use on the automated platform.

This automated assay was validated on 10 hepatocellular carcinomas, 10 IHCC and 20 pancreatic ductal adenocarcinomas.

Quantitative Analysis

The specificity of the signal permitted semiquantitative analysis and the results were recorded as follows:

Cholangiocarcinoma: the percentage of positive tumor cells was recorded at intervals of 5%.

Hepatocellular carcinoma: the tumors were grouped as negative, rare positive (0-10%), focally positive (11-50%), and diffuse positive (>50%).

### Example 1

### Albumin ISH of Normal and Cancer Tissue of the Liver

Intrahepatic cholangiocarcinoma (IHCC) is often a diagnosis of exclusion. Distinguishing IHCC from other metastatic adenocarcinomas based on histopathologic or immunohistochemical analysis is difficult. Therefore, patients are subjected to a high number of procedures and studies in search of a potential primary non-hepatic tumor. Albumin expression is restricted to the liver. Since all liver parenchymal cells are derived from a common embryonic progenitor, the aim of the present study was to confirm that albumin is useful as a biomarker for IHCC, utilizing a novel and highly sensitive RNA in situ hybridization (ISH) platform.

To assess the prevalence of albumin as a specific marker of liver origin, 32 normal livers were stained and demonstrated diffuse positivity for albumin with periportal hepatocytes tending to stain more intensely (see FIG. 6). In all cases, the reactivity was dot-like, each likely representing a single albumin mRNA transcript. No staining was identified in Kupffer cells, endothelial cells, fibroblasts, or stroma. The native bile ducts were negative for albumin, including both large and small caliber ducts. However, reactive bile ductules in the setting of chronic hepatitis and biliary disease were strongly positive for albumin.

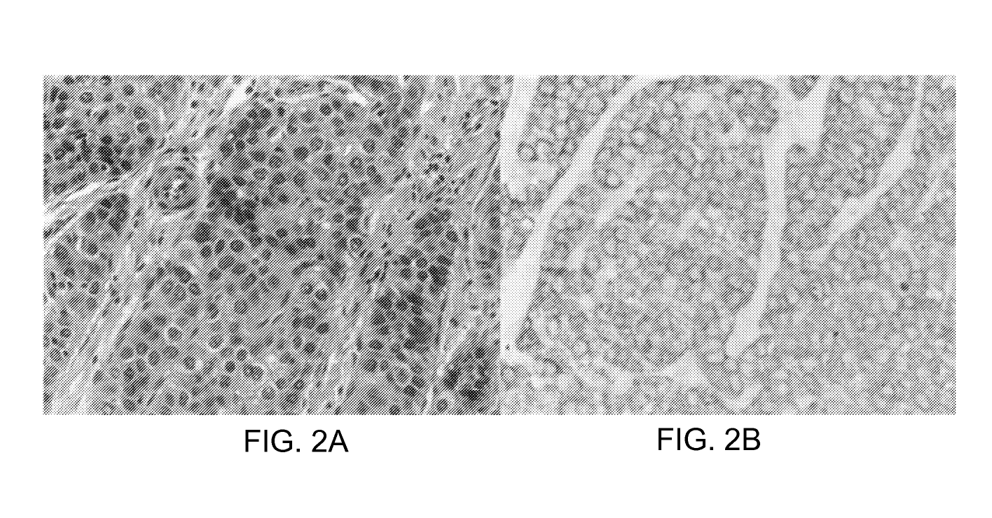

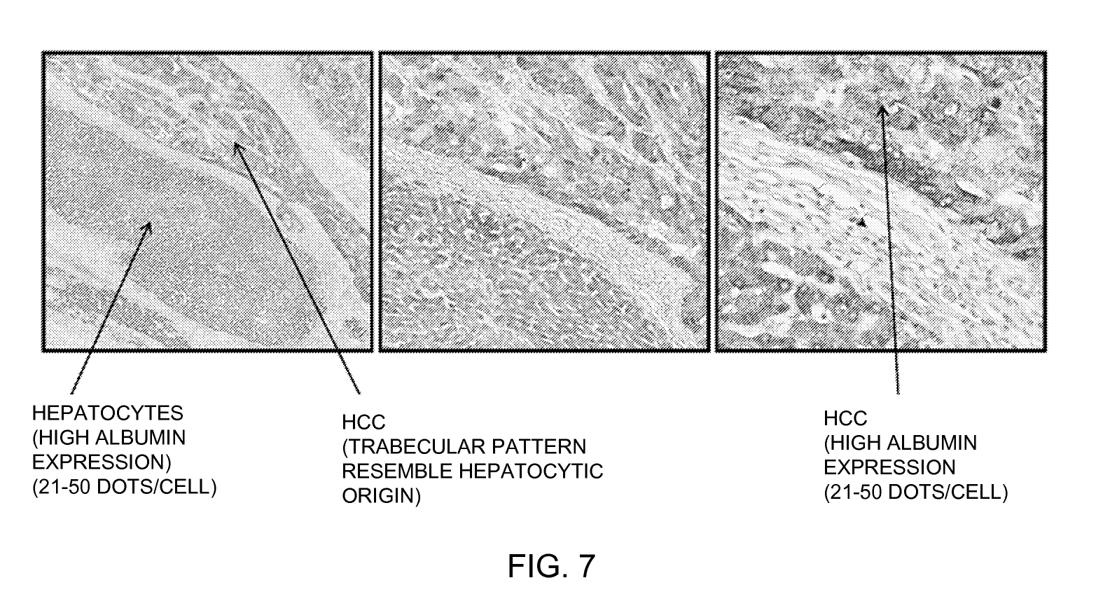

Testing of hepatocellular carcinoma demonstrated 100% staining of all 42 tumors with a diffuse expression pattern (FIGS. 2 and 7). Eight of the tumors were well differentiated, 22 moderately differentiated, and 12 poorly differentiated, yet all demonstrated a diffuse expression pattern, highlighting that albumin is an intrinsic marker of primary liver cancers that is not lost along the spectrum of differentiation.

### Example 2

### Albumin as a Novel Marker for IHCC

A total of 83 resected cases of IHCC were collected from with the mean age of the cohort being 66 years (range 37-86) and 49 patients (59%) were female.

The overwhelming majority of patients presented with a single hepatic lesion (n=66); two cases showed 2 lesions each, while one patient showed multiple intrahepatic lesions. The tumors ranged in size from 1.2 to 10.5 cm (mean 5.5). Histologically, the majority of tumors showed gland formation; among these 8 tumors were classified as well-differentiated and 43 as moderately-differentiated. In 18 tumors, glandular differentiation was only focally observed, constituting <5% of the tumor; these tumors were categorized as poorly differentiated.

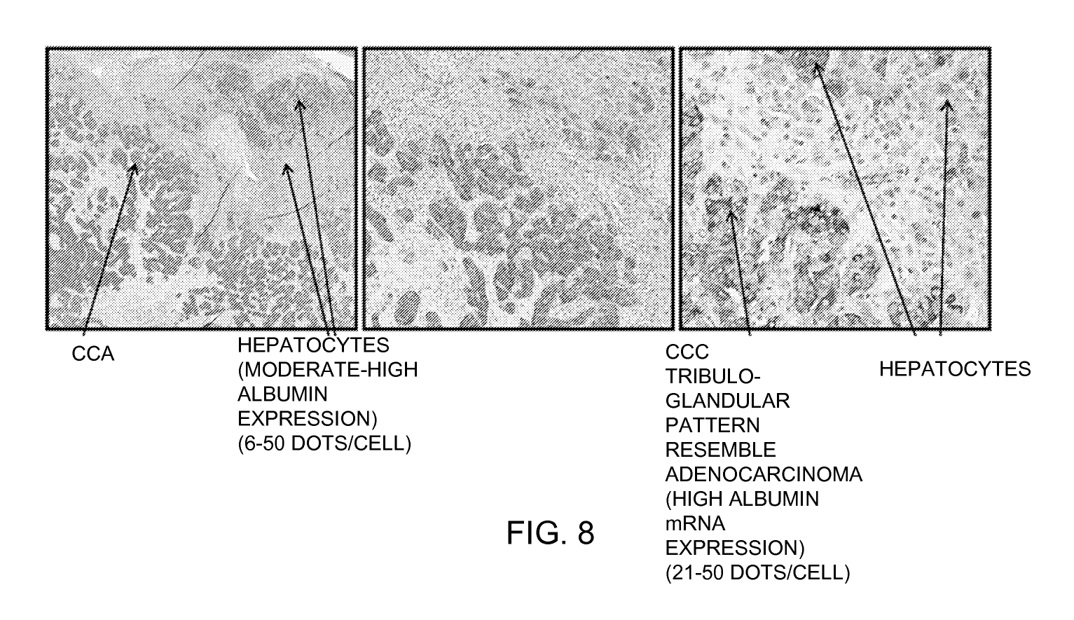

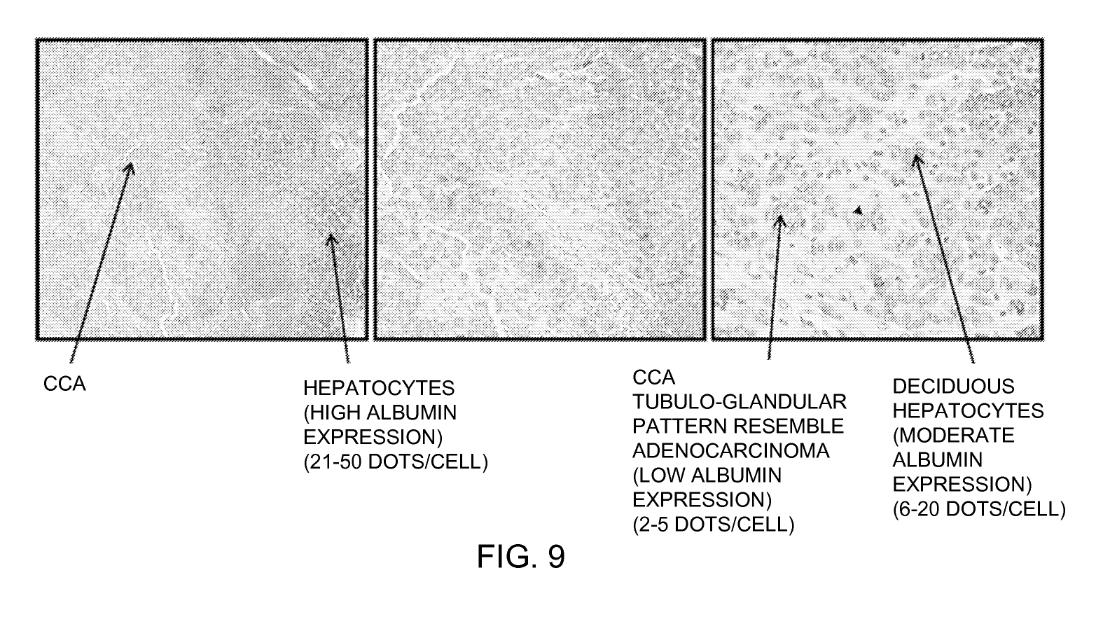

Of the 83 cases, 82 (99%) IHCCs were positive for albumin (FIGS. 3, 8 and 9). The majority of patients (79%) demonstrated reactivity in greater than 50% of the tumor cells. The pattern of reactivity was similar to hepatocellular carcinoma, as the IHCCs demonstrated reactivity in >50% of tumor cells, but notably, the signal intensity per tumor cell in intrahepatic cholangiocarcinomas was less than that seen in hepatocellular carcinomas. There was no evidence of non-specific signal and no staining was identified within stromal or endothelial cells. While all well-differentiated IHCCs were positive, two moderately-differentiated and a single poorly differentiated IHCC was negative for albumin.

Within the positively stained tumors there was heterogeneity of staining where 8% had low (<5% of tumor cells), 25% intermediate (5-50% of tumor cells), and 67% high (>50% of tumor cells) staining frequencies. There was no significant difference in the percentage of positive tumor cells between the 3 grades of IHCC (grade1—84%, grade 2—70% and grade 3—71%, p=0.59).

All 3 mixed hepatocellular-cholangiocarcinomas showed diffuse reactivity in both the hepatocellular and cholangiocarcinoma components.

### Example 3

### Non-Hepatic Adenocarcinoma and Metastatic Tumors to the Liver

Conceptually, bile duct carcinomas appear histologically similar to IHCC, but it is well known that the natural histories of these diseases are quite distinct. Consistent with these differences, albumin RNA ISH was completely negative in both perihilar adenocarcinoma (n=24) and mid-distal bile duct adenocarcinomas (n=22).

Clinically, the most difficult diagnostic problem has been the differentiation of intrahepatic cholangiocarcinoma from metastatic adenocarcinoma to the liver. Therefore, we tested various types of metastatic adenocarcinomas with albumin RNA ISH. Primary adenocarcinomas evaluated included lung (n=22), pancreas (n=210), esophagus and gastroesophageal junction (n=40), stomach (n=72), colon (n=40), bladder (transitional cell carcinoma) (n=8), ovary (n=6), endometrial (n=8), renal cell carcinomas (N=46), and breast (N=34). All tumors were negative for albumin. See FIG. 10.

Intrahepatic metastasis from colon, breast, and lung (n=22) were also examined, in part to investigate the possibility that metastatic tumors to liver may acquire a profile that could mimic a primary hepatic neoplasm. The profile of these adenocarcinomas did not change when they metastasized to the liver: none of them were positive for albumin. However, some metastatic tumors showed infiltrative borders and a meticulous evaluation was often required to distinguish infiltrating tumor cells and non-neoplastic intratumoral bile ductules and hepatocytes, both of which were strongly positive for albumin.

### Example 4

### Evaluation of Tumors of Unknown Origin

Tumors of unknown origin also present a significant clinical dilemma because it is a diagnosis of exclusion and treatment regimens have not been established. A retrospective series of 27 intrahepatic adenocarcinomas whose origin remain uncertain after detailed clinical, radiological, pathologic and immunohistochemical evaluation was evaluated. A total of 6 (22%) of these tumors were positive for albumin on in situ hybridization (Table 1), and therefore were re-classified as IHCC. The other 21 patients (mean age 65 years, 12 males) were negative for albumin. Amongst these latter cases, a clinical/radiologic diagnosis of IHCC was suspected in 7 cases while in 5 cases the histologic and immunohistochemical features raised the possibility of an IHCC, although a wide differential diagnosis including the possibility of metastases was eventually suggested. In the remaining cases the available clinical/radiologic/histologic/immunohistochemical data failed to identify a primary site.

TABLE 1
Clinicopathological details of tumors that were positive for albumin

| | | | Results of | | |
|---|---|---|---|---|---|
| Case | | | immunoperoxidase | Extent of | |
| number | Age | Sex | stains | disease | Diagnosis |
| 1 | 69 | F | Positive: keratin 7, | Multiple liver | Wide differential |
| | | | keratin 19, keratin | lesions, | diagnosis including |
| | | | 20, CA 19.9 | thickening of | intrahepatic |
| | | | Negative: TTF-1, | colon, | cholangiocarcinoma |
| | | | Hep Par 1 | peritoneal | |
| | | | | disease with | |
| | | | | ascites | |
| 2 | 55 | M | Positive: Keratin | Single 8 | Gallbladder |
| | | | 7, keratin 19, | intrahepatic | carcinoma versus |
| | | | keratin 20 | lesion | intrahepatic |
| | | | Negative: PSA, | adjacent to | cholangiocarcinoma |
| | | | PSAP, TTF-1 | gallbladder | |
| 3 | 52 | M | Positive: keratin 7, | Single 9 cm | Favor intrahepatic |
| | | | keratin 19, CA 19 | intrahepatic | cholangiocarcinoma |
| | | | 9 | mass | |
| | | | Negative: keratin, | | |
| | | | 20, CDX2 | | |
| 4 | 87 | F | Negative: Keratin | Multiple | ? Endometrial |
| | | | 7, Keratin 20, | intrahepatic | carcinoma, question |
| | | | TTF-1 | lesions | hepatocellular |
| | | | | | carcinoma |
| 5 | 72 | F | Positive: keratin | Multiple | Primary |
| | | | 19, CA 19-9 | lesions both | intrahepatic |
| | | | Negative: TTF-1 | lobes of liver | cholangiocarcinoma |
| | | | | with | versus metastasis |
| | | | | peritoneal | |
| | | | | implants | |
| 6 | 85 | F | Positive: keratin 7, | Solitary mass | Gallbladder |
| | | | CA 19-9, CA 125, | adjacent to | carcinoma, |
| | | | CDX2 | gallbladder | intrahepatic |
| | | | Negative: keratin | with bile duct | cholangiocarcinoma |
| | | | 20 | stricture | |

### Example 5

### Evaluation of Tumors of Unknown Origin

The two common challenges that face the pathologist evaluating a hepatic neoplasm are: 1) distinguishing a benign from a malignant hepatic neoplasm, and 2) at the poorly differentiated/undifferentiated end of the spectrum, hepatocellular carcinomas often cannot be distinguished from a metastatic adenocarcinoma. There are a wide variety of markers that assist in unraveling the latter diagnostic problem including AFP, polyclonal CEA, CD10, Heppar-1, and Arginase-1.

Amongst these Arginase-1 has emerged as the marker of choice—its high sensitivity combined with the virtual absence of reactivity of non-hepatic neoplasms makes it an almost ideal assay (Yan et al., Am J Surg Pathol 34, 1147-1154 (2010)). Importantly, Arginase-1 also offers a high sensitivity for poorly differentiated hepatocellular carcinomas—86% in a recent analysis (Yan et al., Am J Surg Pathol 34, 1147-1154 (2010)). In that study, Arginase-1 outperformed Hep Par 1 with an overall sensitivity of 96% and 84%, respectively (Yan et al., Am J Surg Pathol 34, 1147-1154 (2010)). Among poorly differentiated hepatocellular carcinomas, however, the two stains may prove complimentary, since a significant percentage of hepatocellular carcinomas negative for Arginase were positive for Hep Par 1.

In this example, albumin bISH (bDNA RNA ISH) was compared to these two leading markers of hepatocellular differentiation—Arginase-1 and Hep Par 1 when used for the diagnosis of HCC in formalin fixed biopsy material.

Materials and Methods

The following materials and methods were used in Example 5.

### Cases

The study cases were selected based on a search of a pathology database.

Hepatocellular Carcinomas

32 cases examples of non-neoplastic liver were examined Tissue microarrays from 76 HCCs were constructed. The arrays were composed of 3 mm cores of paraffin embedded tissue. Moderately and poorly differentiated hepatocellular carcinomas were overrepresented in this cohort. Expression of Arginase-1 and Hep Par 1 were examined on an immunohistochemical platform while that for albumin on a bISH platform (see below for details). Conventional tissue sections from 20 HCCs were also examined for the expression of albumin mRNA. The hepatocellular carcinomas were also graded as well, moderate, and poorly differentiated.

Unclassified Tumors

5 hepatic neoplasms were examined that were classified as undifferentiated carcinomas and lacked histological or immunohistochemical evidence of hepatocellular differentiation. These had previously undergone an extensive immunohistochemical workup. Six additional hepatic neoplasms were examined.

Non-Hepatic Tumors

Albumin expression was examined in a variety of non-hepatic tumors including adenocarcinomas from the lung, esophagus, stomach, colon, pancreas, breast, endometrium, ovary, and transitional cell carcinomas of bladder. In this analysis other mimics of hepatocellular carcinoma were also examined including neuroendocrine tumors of the gastrointestinal tract (n=31), neuroendocrine tumors of the pancreas (n=163) melanoma (n=15), and renal cell carcinoma (n=43). Additionally, 7 acinar cell carcinomas of the pancreas were evaluated.

### Immunohistochemistry

Immunohistochemistry for Hep Par 1 (Dako 1:25; Retrieval EDTA pH 9.0 for 20 mins) and Arginase (Cell Marque 1:600 (Retrieval Citrate pH 6.0 for 30 mins) was performed.

### In Situ Hybridization

Staining with GAPDH was performed as a positive control for mRNA preservation.

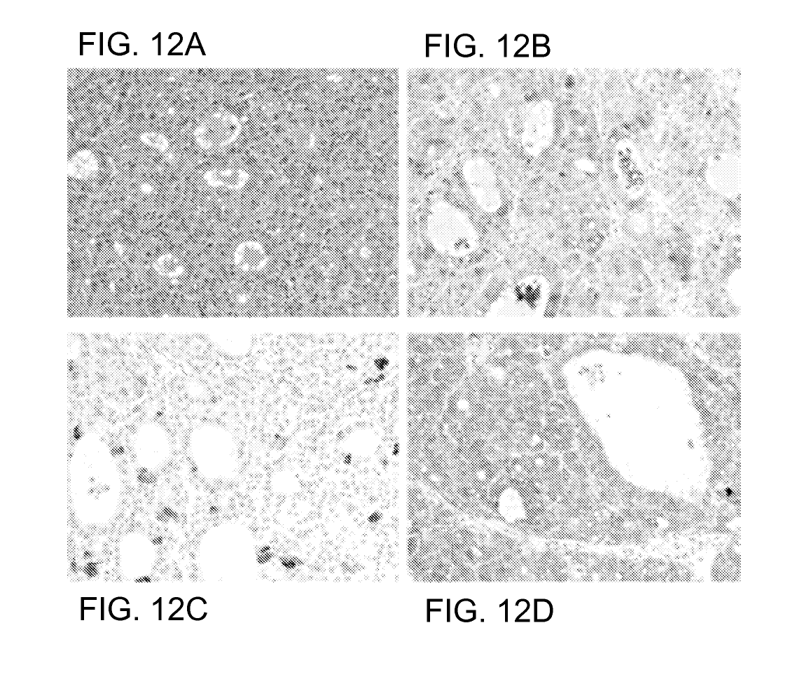

In situ hybridization for albumin was performed using the ViewRNA™ technology (Affymetrix, Santa Clara, Calif.). ViewRNA™ in situ hybridization is based on the branched DNA technology wherein signal amplification is achieved via a series of sequential steps. For the particular assays performed within Example 5, each pair of bound target probe set polynucleotides acts a template to hybridize a pre-amplifier molecule that in turn binds multiple amplifier molecules. In turn, each amplifier molecule provides binding sites to multiple alkaline phosphatase (AP)-conjugated-polynucleotides thereby creating a fully assembled signal amplification “tree” that has approximately 400 binding sites for the AP-labeled probe. Following sequential addition of the fast-red substrate, the AP breaks down the substrate to form a precipitate (red dots) that allows in-situ detection of the specific target RNA molecule (FIG. 12).

In situ hybridization probes (Affymetrix, Santa Clara, Calif.) were designed against albumin transcripts as identified in the NCBI nucleotide database. Briefly, dissected tissues were fixed for <24 hours in 10% Neutral Buffer Formalin at room temperature, followed by the standard formaldehyde-fixed, paraffin-embedded (FFPE) preparation. The FFPE tissues were sectioned at 5+/−1 micron and mounted on Surgipath X-tra glass slide (Leica BioSystems, Buffalo Grove, Ill.), baked for 1 hour at 60° C. to ensure tissue attachment to the glass slides, and then subjected to xylene deparaffinization and ethanol dehydration. To unmask the RNA targets, dewaxed sections were incubated in 500 ml pretreatment buffer (Affymetrix/Santa Clara, Calif.) at 90-95° C. for 10 minutes and digested with 1:100 dilution protease at 40° C. (Affymetrix, Santa Clara, Calif.) for 10 minutes, followed by fixation with 10% formaldehyde at room temperature for 5 minutes. Unmasked tissue sections were subsequently hybridized with 1:50 dilution Albumin probe sets for 2 hours at 40° C., followed by series of post-hybridization washes. Signal amplification was achieved via a series of sequential hybridizations and washes as described in the user's manual. Slides were post-fixed with 4% formaldehyde, counterstained with Gill's hematoxylin, mounted using Dako Ultramount (Dako, Carpinteria, Calif.), and visualized using a standard bright-field microscope. An attempt was made to identify the same three HPFs that were examined on the immunohistochemical platform, and quantification was performed on similar lines.

A semi-quantitative method of scoring was devised. Tumors demonstrating no staining at all were given a score of 0; <5% of cells staining—score 1+, 5% to 50% —score 2+ and more than 50%—score 3. The immunohistochemical stains were graded for intensity: grade 1 and grade 2. The in situ hybridization stain was not graded since the two platforms could not be compared. However, based on prior experience with the technology, any dots in excess of 1 per 100 cells is considered positive.

Results

Demographics

The mean age of the cohort of patients with hepatocellular carcinoma was 69 years (standard deviation 11) with 55 males and 21 females.

### Normal Liver and Background Cirrhosis

In all 32 cases examined normal hepatocytes were diffusely positive for albumin. The periportal hepatocytes stained more intensely than zone 3 hepatocytes.

The cirrhotic nodules were diffuse positive for albumin and no regional variations were noted. The background liver showed cirrhosis in 70 of the 75 cases.

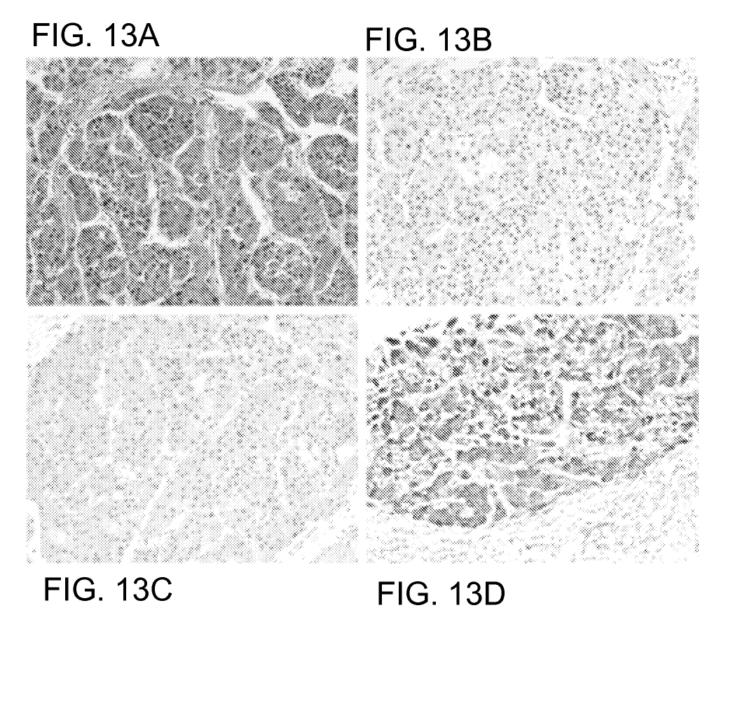

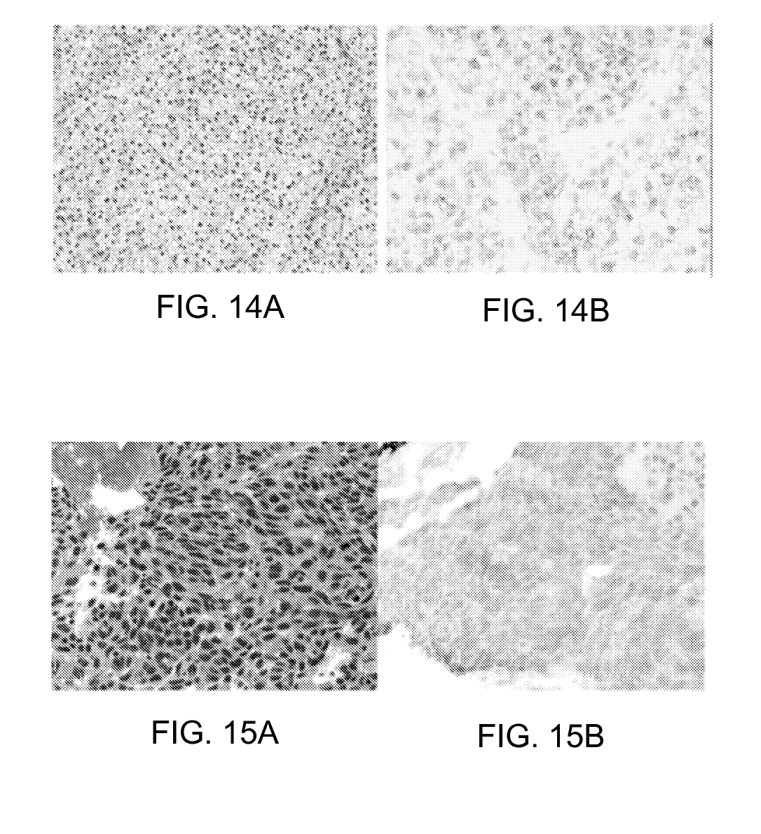

Albumin—74 of 76 HCCs were positive for albumin (FIGS. 12, 13 and 14). One of the tumors negative for albumin failed GAPDH and was not included in the final analysis. The suboptimal mRNA preservation was likely related to radiofrequency ablation. Among the positive cases, 72 cases showed reactivity in >50% of the tumor, and all 74 cases showed characteristic dot like reactivity in >5% of the tumor cells. All 3 examples of clear cell variants of hepatocellular carcinoma were positive for albumin with >50% of the tumor positive is all 3 cases.

Hep Par 1—68 of the 75 HCCs were positive for Hep Par1. The sensitivity of the assay was 91%. 5 of the 68 (7%) positive cases showed weak (1+) reactivity.

Arginase-1—65 of the 75 HCCs were positive for Arginase-1. The sensitivity of the assay was 87%. 19 (29.2%) of the tumors that were positive for Arginase-1 were weakly (1+) positive.

Comparison of Albumin with Hep Par 1 and Arginase-1

The only hepatocellular carcinoma (moderately differentiated) that was negative for albumin was positive for both Hep Par 1 and Arginase-1. Among the 7 tumors negative for Hep Par 1, 3 were positive for Arginase-1 and among the 10 tumors negative for Arginase-1, 5 were positive for Hep Par 1.

### Automated Platform

15 hepatocellular carcinomas (whole sections) were examined for albumin. All 15 cases were diffusely positive (greater than 95%). Six additional hepatocellular carcinomas from 2 institutions were also evaluated, all of which were diffusely positive for albumin.

### Specificity of Albumin as a Marker of Hepatocellular Differentiation

Adenocarcinomas from the lung (n=22), pancreas (n=95), esophagus and gastric-cardia (n=40), stomach (n=72), colon (n=40), transitional cell carcinoma (n=8), ovary (n=6), and endometrial adenocarcinoma (n=8) were negative for albumin.

Cases were evaluated that could potentially mimic hepatocellular carcinomas, including melanoma, renal cell carcinomas and pancreatic endocrine neoplasms, all of which were negative for albumin. However, 2 of 7 acinar cell carcinomas were positive for albumin.

### Difficult to Characterize Hepatic Neoplasms

Of the 5 undifferentiated tumors, 3 were positive for albumin (FIG. 15). One of the cases negative for bISH was eventually diagnosed as gallbladder carcinoma. The biopsy from case 3 was composed of mostly necrotic tissue and the few viable cells were negative for albumin. Based on the presence of cirrhosis and the markedly elevated levels of AFP a diagnosis of hepatocellular carcinoma was eventually favored.

All five neoplasms were negative for Hep Par 1; however, 1 of the 3 tumors was positive for Arginase-1.

### DISCUSSION

The present results suggest that albumin bISH is superior to Arginase-1. A higher percentage of tumor cells were positive for albumin. Additionally, almost a third of the tumors that were positive for Arginase-1 showed only faint reactivity for this protein. In our experience this low intensity reactivity is often only equivocal evidence of hepatocellular differentiation. The strongly positive red dot-like staining pattern makes the bISH assay easier to interpret than immunohistochemistry. The interpretation was further simplified by the virtual absence of non-specific signal. It should be noted that this analysis was performed on routinely processed paraffin embedded tissue and there were no specific efforts to ensure preservation of RNA.

Based on this and prior data the specificity of bISH for albumin and immunohistochemistry for Arginase-1 is roughly equivalent. Only rare non-hepatic tumors stain positively for Arginase-1—a single prostatic adenocarcinoma (Yan et al., Am J Surg Pathol 34, 1147-1154 (2010)). The increased sensitivity does not lead to a loss of specificity and a diverse group of carcinomas including those arising from the lung, breast, esophagus, stomach, colon, ovary, urinary bladder lacked reactivity for albumin.

Prior efforts at detecting albumin in hepatic neoplasms used digoxin labeled oligonucleotide probes to label albumin mRNA and did not employ amplification (Kakar et al., American Journal of Clinical Pathology 119, 361-366 (2003); Oliveira et al., Am J Surg Pathol 24, 177-182 (2000); Krishna et al., Am J Surg Pathol 21, 147-152 (1997); Murray et al., J Clin Pathol 45, 21-24 (1992); D'Errico et al., Hum Pathol 27, 599-604 (1996); D'Errico et al., Diagn Mol Pathol 7, 289-294 (1998); Yamaguchi et al., Virchows Arch B Cell Pathol Incl Mol Pathol. 64, 361-365; Wood et al., Journal of Cutaneous Pathology 36, 262-266 (2009)). Nevertheless, the sensitivity of the assay ranged from 93-95% (Kakar et al., American Journal of Clinical Pathology 119, 361-366 (2003); Oliveira et al., Am J Surg Pathol 24, 177-182 (2000); Krishna et al., Am J Surg Pathol 21, 147-152 (1997)). Similar to this analysis, there was no loss of sensitivity with poorly differentiated hepatocellular carcinomas and clear cell carcinoma.

The bDNA RNA ISH assay used in the present example significantly increased the sensitivity of the assay and was capable of detecting copy numbers as low as 1-2 per cell. As is evident from the studies using traditional in situ hybridization platforms, amplification is not critical for detecting albumin in some hepatocellular carcinomas, which express high levels of albumin. However, in approximately 5% of hepatocellular carcinomas albumin is expressed in low levels requiring an amplification technique to uncover its presence, thus accounting for the almost perfect sensitivity of the current assay.

The present findings were validated by evaluating a cohort of poorly differentiated/undifferentiated carcinomas of the liver. These five cases, identified over a 5-year period, lacked unequivocal histological or immunohistochemical evidence of hepatocellular differentiation (although in retrospect Arginase-1 was positive in 1 of 3 cases evaluated). Three of the five cases were strongly (100% of cells stained) positive for albumin. In retrospect, one case represented a gallbladder carcinoma, and was negative for albumin.

Other potential mimics of hepatocellular carcinoma, including melanoma, renal cell carcinoma and neuroendocrine tumors, were negative for albumin. The significance of reactivity in two of the seven acinar cell carcinomas evaluated is uncertain.

The bISH platform used in the present example offers pathologists a novel means of detecting mRNA, including products that are expressed at low copy numbers. The availability of this technology is particularly valuable for detecting secreted proteins such as albumin as well as targets against which an immunohistochemical approach has been unsuccessful. The bright red dots (from the use of fast red with the alkaline phosphatase label probes) generated by this technology make the assessment of these preparations straightforward. It should be emphasized that the presence of even rare dots (>3 per cell) is strong evidence of a positive signal since non-specific signal is minimal—in our experience <1 dot per 10 cells.

To conclude, in situ hybridization offers a robust means of detecting hepatocellular differentiation, superior to all currently available platforms. The ability to automate the in situ hybridization process opens up the prospect of its use in the routine diagnostic pathology laboratory.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining the origin of a tumor in a subject, the method comprising:
contacting a sample comprising cells or tissue from the tumor with a plurality of polynucleotide probes that bind specifically to albumin mRNA in situ;
detecting binding of the probes to albumin mRNA, and
identifying a sample in which the probes bind to albumin mRNA as a tumor of hepatic origin, or identifying a sample in which the probes do not bind to albumin mRNA as a tumor of nonhepatic origin.

2. A method of selecting a treatment for a subject who has a tumor, the method comprising:
contacting a sample comprising cells or tissue from the tumor with a plurality of polynucleotide probes that bind specifically to albumin mRNA in situ;
detecting binding of the probes to albumin mRNA, and
identifying a sample in which the probes bind to albumin mRNA as a tumor of hepatic origin, and selecting for the subject a treatment for a hepatic tumor; or
identifying a sample in which the probes do not bind to albumin mRNA as a tumor of nonhepatic origin; determining the tissue of origin of the tumor; and
selecting for the subject a treatment for a cancer of the tissue of origin.

3. A method of treating a subject who has a tumor, the method comprising:
contacting a sample comprising cells or tissue from the tumor with a plurality of polynucleotide probes that bind specifically to albumin mRNA in situ;
detecting binding of the probes to albumin mRNA, and
identifying a sample in which the probes bind to albumin mRNA as a tumor of hepatic origin, and administering to the subject a treatment for a hepatic tumor; or
identifying a sample in which the probes do not bind to albumin mRNA as a tumor of nonhepatic origin; determining the tissue of origin of the tumor; and
administering to the subject a treatment for a cancer of the tissue of origin.

4. The method of claim 1, further comprising determining whether a tumor of hepatic origin is Hepatocellular Carcinoma (HCC) or Intrahepatic Cholangiocarcinoma (IHCC), or determining whether a tumor of hepatic origin is HCC, IHCC, or a bile duct adenoma (BDA).

5. The method of claim 4, wherein whether the tumor is HCC or IHC or BDA is determined based on morphology of the tumor cells in the sample, wherein a trabecular arrangement of tumor cells resembling normal hepatocytes indicates the presence of HCC, wherein a tubulo-glandular arrangement of tumor cells resembling adenocarcinoma indicates the presence of IHCC, and wherein tumor architecture resembling adenoma with a well differentiated glandular pattern indicates the presence of BDA.

6. The method of claim 1, further comprising one or more of:
identifying a sample in which the probes bind to albumin mRNA, and wherein there is a gradient of albumin mRNA present in hepatoctyes with low to moderate expression in Zones 1 and 3 and high expression in Zone 2, as comprising normal liver;
identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma;
identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma;
identifying a sample in which the probes bind to albumin mRNA, there are low levels of albumin mRNA expression, and tumor architecture resembling adenoma with a well differentiated glandular pattern as comprising BDA; and
identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease.

7. The method of claim 2, further comprising one or more of:
identifying a sample in which the probes bind to albumin mRNA, and wherein there is a gradient of albumin mRNA present in hepatoctyes with low to moderate expression in Zones 1 and 3 and high expression in Zone 2, as comprising normal liver, and not treating the subject;
identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma (HCC), and selecting a treatment for HCC for the subject;
identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma (IHCC), and selecting a treatment for IHCC for the subject;
identifying a sample in which the probes bind to albumin mRNA, there are low levels of albumin mRNA expression, and tumor architecture resembling adenoma with a well differentiated glandular pattern as comprising BDA, and selecting a treatment for BDA for the subject; and
identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease, and selecting a treatment for the primary cancer for the subject.

8. The method of claim 3, further comprising one or more of:
identifying a sample in which the probes bind to albumin mRNA, and wherein there is a gradient of albumin mRNA present in hepatoctyes with low to moderate expression in Zones 1 and 3 and high expression in Zone 2, as comprising normal liver, and not treating the subject;
identifying a sample in which the probes bind to albumin mRNA and wherein there is moderate to high levels of albumin mRNA expression, and tumor architecture that is recognizable as hepatocytic in origin with a trabecular pattern based on histopathological analysis, as comprising Hepatocellular Carcinoma (HCC), and administering a treatment for HCC to the subject;
identifying a sample in which the probes bind to albumin mRNA and wherein there is a range of albumin mRNA expression, and tumor architecture that resembles adenocarcinoma with a tubulo-glandular pattern based on histopathological analysis, as comprising Intrahepatic Cholangiocarcinoma (IHCC), and administering a treatment for IHCC to the subject;
identifying a sample in which the probes bind to albumin mRNA, there are low levels of albumin mRNA expression, and tumor architecture resembling adenoma with a well differentiated glandular pattern as comprising BDA, and administering a treatment for BDA to the subject; and
identifying a sample in which the probes do not bind to albumin mRNA in the tumor cells, wherein there is adjacent normal tissue shows albumin expression, and/or tumor architecture that resembles a pattern from a primary cancer, as comprising metastatic liver disease, and administering a treatment for the primary cancer to the subject.

9. A method of making a differential diagnosis between metastatic liver disease and a primary tumor of hepatic origin in a subject who has a tumor, the method comprising:
contacting a sample comprising tissue from the tumor with a plurality of polynucleotide probes that bind specifically to albumin mRNA in situ;
detecting binding of the probes to albumin mRNA, and
identifying a sample in which the probes bind to albumin mRNA as a tumor of hepatic origin, or
identifying a sample in which the probes do not bind to albumin mRNA as a tumor of nonhepatic origin.

10. The method of claim 1, wherein the sample is from a tumor that is in the liver of the subject.

11. The method of claim 1, wherein the sample is from a tumor that is not in the liver of the subject.

12. The method of claim 1, wherein the plurality of probes comprises probes that bind to a plurality of target regions in the albumin mRNA.

13. The method of claim 1, wherein the binding of the probes to albumin mRNA is detected using branched nucleic acid signal amplification.

14. The method of claim 13, wherein the probes are branched DNA probes.

15. The method of claim 14, comprising contacting the sample with a plurality of probes that comprises one or more label extender probes that bind to a plurality of target regions in the albumin mRNA; hybridizing one or more pre-amplifier probes to the one or more label extender probes; hybridizing one or more amplifier probes to the pre-amplifier probes; and hybridizing one or more label probes to the one or more amplifier probes.

16. The method of claim 15, wherein the label probe is conjugated to alkaline phosphatase (AP), and binding of the probe is detected using fast red or fast blue as a substrate for the alkaline phosphatase.

17. The method of claim 1, wherein the sample is a biopsy sample obtained from the subject.

18. The method of claim 1, wherein the sample is a formaldehyde-fixed, paraffin-embedded (FFPE) clinical sample.

19. The method of claim 1, wherein the tissue comprises a plurality of individually identifiable cells.

20. The method of claim 1, further comprising:
contacting a sample comprising tissue from the tumor with a plurality of polynucleotide probes that bind specifically to mRNA encoding a housekeeping gene (HKG) in situ;
detecting binding of the probes to HKG mRNA, and
selecting for further analysis a sample in which binding of probes to the HKG mRNA is detected, or rejecting a sample in which binding of probes to the HKG mRNA is not detected.

21. The method of claim 20, wherein the binding of the probes to albumin mRNA or HKG mRNA is detected using branched nucleic acid signal amplification.

22. The method of claim 21, wherein the probes are branched DNA probes.

23. The method of claim 22, comprising contacting the sample with a plurality of probes that comprises one or more label extender probes that bind to a plurality of target regions in the albumin or HKG mRNA; hybridizing one or more pre-amplifier probes to the one or more label extender probes; hybridizing one or more amplifier probes to the pre-amplifier; and hybridizing one or more label probes to the one or more amplifier probes.

24. The method of claim 23, wherein the label probe is conjugated to alkaline phosphatase (AP), binding of the albumin probes to albumin mRNA is detected using fast red as a substrate for the alkaline phosphatase, and binding of the HKG probes to HKG mRNA is detected using fast blue as a substrate for the alkaline phosphatase.

25. A kit for performing the method of claim 1, wherein the kit comprises a plurality of polynucleotide probes that bind specifically to albumin mRNA in situ comprising:
i. one or more label extender probes that bind to a plurality of target regions in the albumin mRNA;
ii. one or more pre-amplifier probes that are capable of hybridizing to the one or more label extender probes;
iii. one or more amplifier probes that are capable of hybridizing to the pre-amplifier probes; and
iv. one or more label probes that are capable of hybridizing to the one or more amplifier probes.

26. The kit of claim 25, wherein the kit further comprises a plurality of polynucleotide probes that bind specifically to mRNA encoding a housekeeping gene (HKG) in situ.


SEQUENCE LISTING (1 sequence)

SEQ ID NO: 1 - DNA, 2264 bp, Homo sapien
agtatattagtgctaatttccctccgtttgtcctagcttttctcttctgtcaaccccacacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaaaaaaaaaaaaaaaaaaa